(19) United States
(12) Patent Application Publication
(10) Pub. No.: US 2009/0068126 A1
(43) Pub. Date: Mar. 12, 2009

(54) Composition for surface photoprotection

(76) Inventors: Luiz Antonio Soares Romeiro, Aguas Claras Norte Taguatinga (BR); Viviane Candida da Silva, Goiania/Goias (BR); Maria Marcia Murta, Sobradinho (BR); Gouvan Cavalcante Magalhaes, Asa Norte (BR); Lucio Paulo Lima Logrado, Asa Norte (BR); Maria Lucilia dos Santos, Asa Norte (BR); Ines Sabioni Resck, Octogonal (BR); Emeli de Araujo Moura, Rio de Janeiro (BR); Sheila Garcia, Rio de Janeiro (BR); Gisela Maria Dellamora-Ortiz, Rio de Janeiro (BR); Alvaro Augusto da Costa Leitao, Rio de Janeiro (BR); Celia-Santos da Silva, Rio de Janeiro (BR); Zaida Maria Faria de Freitas, Rio de Janeiro (BR); Elisabete Pereira dos Santos, Rio de Janeiro (BR)

Correspondence Address: Carter, DeLuca, Farrell & Schmidt, 445 Broad Hollow Road, Suite 225, Melville, NY, 11747, US

(21) Appl. No.: 11/665,815

(22) PCT Filed: Oct. 21, 2005
(86) PCT No.: PCT/BR05/00221

§ 371 Date: Apr. 19, 2007

(30) Foreign Application Priority Data
Oct. 21, 2004 (BR) P1040.6040-7

(51) Int. Cl. A61K 8/36; C07C 65/03; A61Q 17/04; C07C 39/04

(52) U.S. Cl. 424/59; 562/478; 568/716

(57) ABSTRACT

The molecules capable of absorbing ultraviolet radiation from the cashew nut shell liquid changes are the object of the present invention; it is also described the compositions responsible for protecting the surfaces and chemical processes for the referred molecules production.

### FIELD OF INVENTION

The present invention is related to compounds capable of absorbing ultraviolet radiation, more specifically the non-isoprenoid phenol derivatives that can be obtained from the cashew nut shell liquid. Its use in many compositions and the processes for obtaining them are described here.

### DESCRIPTION OF THE RELATED ART

The Sun emits a wide spectrum of electromagnetic radiation, which is transmitted through the space in the form of waves. In a general way, the composition of the solar spectrum in the terrestrial surface is of approximately 10% of ultraviolet radiation (290-400 nm), 49% of visible light (400-760 nm) and 45% of infrared radiation (760-3000 nm). In spite of small, the portion of ultraviolet radiation is responsible for 99% of the undesirable effects of the incident solar light on the surface of the Earth. According to its physical properties and biological effects, the wavelength of ultraviolet radiation (200-400 nm) can be divided in sub-regions: UV-C (200-280 nm), UV-B (280-320 nm) and UV-A (320-400 nm), being this last one still subdivided in UV-AI (320-360 nm) and UV-AII (360-400 nm).

The substances differ in their tendencies of absorbing light of a given wavelength, in function of the involved chemical species structures and of the different energy levels of their electrons. The molecules of oxygen don't absorb radiation in the visible wavelength of the electromagnetic spectrum (400-760 nm), but they tend to absorb it in the ultraviolet (UV) wavelength range among 125-175 nm, with the maximum absorption at, approximately, 140 nm. In this context, the presence of molecular oxygen in the stratosphere and above it is responsible for the absorption of the solar light in the UV range of 120-200 nm, while the radiation in the wavelength of 220-320 nm is absorbed by molecules of ozone (O₃), that are dispersed in the medium and lower stratosphere. Acting synergistically, the molecules of O₂ and O₃ absorb the whole ultraviolet radiation in the wavelength of 220-290 nm, which superpose UV-C (200-280 nm). Although molecules of O₃ can absorb radiations in the UV-B wavelength (280-320 nm), their capacity is limited in this radiation wavelength, being dependent of the latitude, making possible ca 10 to 30% of this radiation type to reach the Earth's surface. In this way, the layer of ozone is not completely effective in the alive beings' protection regarding the UV-B radiation, once the absorption for O₃ fails in an almost exponential way for this radiation wavelength. The reduction of the stratospheric ozone concentration allows a larger amount of ultraviolet light (UV-B) to reach the Earth's surface, which decrease of 1% in this natural protection layer results in an increase of about 2% in the radiation intensity in the fundamental level.

Ninety-five percent of the UV rays that reach the Earth are UV-A and only 5% are UV-B. The UV-B rays are more intense from 11:00 A.M. to 3:00 P.M. during the summer and, due to their energy, they penetrate the derme (superficial layer of the skin) resulting in biological consequences as human skin burns, whose overexposition can lead to skin cancers, affect the immunological system, the animals and the growth of some plants. Starting from the variation in the UV-B wavelength band, it was verified that the most harmful effect happens with a light absorption of about 300 nm, responsible for most of the skin carcinomas, which incidence of malignant melanoma is related to short exhibition periods to the high energy UV light, particularly in populations with smaller melanin content.

Considering the least energetic ultraviolet range (UV-A), this radiation region (320-400 nm) is not significantly absorbed neither by molecules of ozone nor by any constituent of the non-polluted atmosphere. Most of this UV-type radiation surpasses the natural barriers of absorption and reaches the terrestrial surface. The UV-A rays are present 24 hours everyday of the year, being dangerous both in the summer and winter. Particularly, this radiation doesn't burn nor lead to red skin, but it is related to the premature aging of the skin, whose cumulative effect provokes stains and wrinkles. Recently it was discovered that the UV-A rays make way for UV-B ones, potentiating its action in the appearance of cancerous cases. (Veiga, A., 2002, Salve sua pele, Revista Época, 237:84-91).

The solar burn consists of the cutaneous inflammation that most of the people experiences after a large exposition to the solar radiation, being characterized by skin redness, pain or hypersensitivity, edema and, in extreme cases, by the formation of bubbles and the skin detachment. The erythematous reaction is transitory, usually expressing itself within some minutes or hours after the exposition, reaching its peak in 12 or 14 hours and being persistent for several days. The intensity of this response depends on each individual's skin sensibility to the Sun and on the amount of absorbed energy. The damage caused to the skin because of Sun exposure is not limited to the solar burn, being cumulative and, probably, irreversible, being able to produce alterations on the collagenous and elastic fibers, as well as some loss of subcutaneous adipose tissue. This premature aging process continues when, as time goes by, there is an increase of the exposition to the solar radiation.

Epstein (J. Am. Acad. Dermatol. 1983, 9: 487-504) developed some works on skin pathologies induced by the light e.g. cutaneous degeneration, photosensitization and phototoxicity, as well as on cutaneous diseases induced by the UV light e.g. wrinkleness of skin, atrophies and actinic keratosis. It can be seen in these works that, even with evidences of benign formation of keratotic plaques, the actinic elastosis is seen as a precancerous condition deserving attention as for the diagnosis. Of the several types of skin cancers, only the squamous cell carcinoma was evidenced in studies with animals exposed to the UV light, being this malignancy type found in the individuals of white skin face (Caucasian), corroborating evidences from the studies with mice. However, no evidence related to the light impact in the basocellular carcinoma was verified, although its distribution i.e. head, neck and hands, seems to indicate the participation of the light induction (Cancer of the skin. American Academy of Dermatology, Evanston, Ill., 1985).

The action spectrum related to the carcinogenesis seems to coincide with the one related to the erythema i.e. 290-320 nm (Pathak, M. A., J. Am. Acad. Dermatol. 1982, 7: 285-311), having evidences that the exposition to UV-A can predispose the adverse effects of UV-B (Strickland, P. T., J. Invest. Dermatol. 1986, 87: 272-275). It is accepted, in the literature, that the UV light not only reduces the number as well as it harms the epidemic Langerhans cells (LC) preventing them from recognizing the hapten and stimulating the effector via of the immune response (I Erase, V. A., Dawes, L. & Jackson, R., J. Invest. Dermatol. 1981, 76: 330-331; Nussbaum, B. P., Edwards, E. K., Horwitz, S. N. & Frost, P., Ach Dermatol., 1983, 119: 117-121). Additionally, it was postulated that the UV-B radiation determines the formation of T-lymphocytes suppressors that interfere in the rejection of cutaneous cancers induced by that radiation, indicating that the UV light not only harms DNA of the skin, but it interferes in its capacity to destroy this lesion through the immunological system. Other undesirable effects of the UV light exposition are related to the development, in vivo, of the ornithine decarboxylase enzyme (ODC) in mice (Kligman L. H. & Kaidbey, K. H., Photochem Photobiol. 1986, 43: 649-654), which participates in the polyamines formation, responsible for the induction of the cellular proliferation, as well as in the histamine liberation through induction of the Ehrlich ascite cells, activated by the irradiation of the pheomelanin through UV-A light (Ranadive N. S., Shirwadkar, S., Persad, S. & Menon, I. A., J. Invest. Dermatol. 1986, 86:303-307).

As for the processes of light-molecules interaction, the absorption of energy, whose wavelength corresponds to the visible or ultraviolet region, usually results from the excitement of bonding and nonbonding electrons, consequently, the wavelength of the absorption peak can be correlated with the connection type in the species in study (Skoog, D. A., Holler, F. J., Nieman, T. A., 1998, Principles of instrumental analysis, 5a ed., Saunders, College Publishing, USES). There are three different classification for the electrons in a molecule: a) electrons in covalent bond (bond σ) are strongly linked, and to excite them it is necessary high energy radiation (small wavelength); b) electrons bond to atoms such as the ones from chlorine and oxygen, through an isolated pair are nonbonding (n) and they can be excited with smaller energy (larger wavelength) than the bonding electrons; c) electrons in double or triple bonds (electrons π) can be somehow, easily excited In the molecules with alternate double bonds (conjugated systems), the electrons π are delocalized and demand less energy for excitement, so that the absorption moves for the larger wavelengths (Jeffery, G. H., Bassett, J., Mendham, J., Denney, R. C., Vogel, A., 1992 Quantitative Chemical Analysis, 5a ed., Publisher Guanabara Koogan, Rio de Janeiro).

All the organic compounds are capable of absorbing electromagnetic radiation because all of them contain valency electrons that can be excited for higher levels of energy. The absorption of ultraviolet and visible radiation in the larger wavelength region is restricted to a limited number of functional groups (called chromophores) that contain valency electrons with relatively low excitement energy. (Skoog, D. A., Holler, F. J., Nieman, T. A., 1998, Principles of instrumental analysis, 5a ed., Saunders, College Publishing, USES). These groups invariably contain double or triple bonds and they include the nitro, nitroso, azo, carbonyl and thiocarbonyl groups. If there is chromophore conjugation with the same species, or from different species, a new absorption band will appear in a larger wavelength (Jeffery, G. H., Bassett, J., Mendham, J., Denney, R. C., Vogel, A., 1992 Quantitative Chemical Analysis, 5a ed., Publisher Guanabara Koogan, Rio de Janeiro). The absorption of a given molecule can also be enhanced by the presence of the denominated auxochrome groups e.g. OH, NH₂, CH₃ e NO₂, which don't absorb significantly in the ultraviolet area, but that have deep effect on the absorption of the molecule in which they are bonded to. The auxochromes (substituents) have, at least, a pair of nonbonding electrons “n” capable of interacting, for instance, with the electrons π of a benzenic ring. This interaction has the effect of stabilizing the state π*, lowering its energy, resulting in a bathochromic displacement i.e. moving the absorption peak for the larger wavelengths (Skoog, D. A., Holler, F. J., Nieman, T. A., 1998, Principles of Instrumental Analysis, 5a ed., Saunders, College Publishing, USES; Jeffery, G. H., Bassett, J., Mendham, J., Denney, R. C., Vogel, A., 1992 Quantitative Chemical Analysis, 5a ed., Publisher Guanabara Koogan, Rio de Janeiro). The inverse effect, in other words, the displacement of the absorption peak for smaller wavelengths i.e. hipsochromic effect, is related, usually, to substitutions or effects of the solvent.

The absorption is quantitatively treated by the Lambert-Beer Law, which infers about the fraction of monochrome light transmitted through an absorbent system and it is expressed through the relationship:

Iₜ/Iₒ=10^{εbc}=e^{εbc}

or

A=−log T=log Pₒ/P=εbc

where Iₜ is the light intensity after it passes through the sample and Iₒ is the initial light intensity, A is the absorbance, c is the absorbent concentration, b is the optical pathlength (cm), through which the light travels, and ε is the absorptivity coefficient. According to this law, the concentration c of an absorbent analyte is linearly related with the absorbance (Skoog, D. A., Holler, F. J., Nieman, T. A., 1998, Principles of Instrumental Analysis, 5a ed., Saunders, College Publishing, USES; Wayne, C. E., Wayne, R. P., 1996 Photochemistry, Oxford Chemistry Primers, In the 39, New York). The molar absorptivities (ε) can vary from zero to 10⁵ and they are observed in the molecular absorption of the ultraviolet or visible light, whose allowed transitions present strong absorption bands (ε_{max.}=from 10⁴ to 10⁵), as well as molar absorptivity peaks smaller than 10³ are classified as low intensity, resulting from forbidden transitions (Skoog, D. A., Holler, F. J., Nieman, T. A., 1998, Principles of Instrumental Analysis, 5a ed., Saunders, College Publishing, USA).

The protection against the UV light is related to the reduction in the exposition time to the UV rays, and it can be obtained by the application of sunscreens. The commercially available ones are based on two principles: scattering or absorption of the radiation, corresponding to two categories of protecting agents: inorganic and organic, respectively.

The inorganic sunscreens are known as physical, mineral, insoluble, natural or non-chemical. During the last decade the inorganic sunscreens have more frequently been used in activities in the Sun and for daily photoprotection. This is due partly to their safety and effectiveness, particularly as UV-A blocking. The most used ones are made of titanium dioxide (TiO₂) and the zinc oxide (ZnO). Those compounds exist as white powder, inodorous and they are sufficiently opaque to reflect and scatter the incident radiation. The particles size is, approximately, 0.20 μm or smaller, and each particle has visible light maximum scattering related to its size (Gasparro, P. F., Mitchnick, M., Nash, F., Photochem. Photobiol. 1998, 68: (3) 243-256)

The organic sunscreens are referred to as soluble or chemical and their structure is similar concerning to the presence of aromatic compounds substituted with high conjugation degree. To be well acquainted with how the organic sunscreens work, the interaction between molecule and light must be understood. The light absorption for the molecule is associated with the chromophore part of its structure. For organic molecules, the responsible chromophore for the UV light absorption is usually associated to the electron π displacement in conjugated systems. Generally, when a molecule absorbs a photon whose energy is sufficiently high, an electron is promoted from a lower level of energy to a higher level of energy and it's said that the molecule goes from the ground to the excited state. The most common state for an organic molecule is the first singlet excited state, in which the promoted electron has a paired spin. In the excited state, the molecule has many ways of losing this energy: a) the molecule can emit a photon and return to the ground state (fluorescence); b) the molecule can return to the ground state for the emission of thermal energy through a series of transitional vibration (vibrational relaxation or non-radioactive decay); c) the molecule can suffer some kind of reaction in the excited state; d) the molecule can convert its energy to an excited state of smaller energy, triplet state, in which the electrons are unimpaired. This excited state can return to the ground state through radioactive (phosphorescence) or non-radioactive (vibrational decay) processes or it can suffer photochemical reactions. What will be done is going to depend on the relative speed of each process, and the one that presents larger speed, which depends on the choromophore nature and on the molecule structure, has an advantage (Kimbrough, D. R., J. Chem. Ed., 1997, 74: (1) 51-53).

In a general way, those photoabsorbent agents work absorbing radiation in the UV region, followed by a very fast vibrational relaxation to the ground state. Once in the ground state those molecules can absorb another light photon, repeating the process and, so, protecting the skin from the UV radiation. Any molecule whose vibrational relaxation to the ground state consists of the fastest way of distributing energy from the excited state can act as sunscreen (Kimbrough, D. R., J. Chem. Ed., 1997, 74: (1) 51-53).

Among the main commonly used organic sunscreen in commercial formulations there are the derived from the p-aminobenzoic acid (PABA) e.g. ethyl hydroxypropyl aminobenzoate, glyceryl p-aminobenzoate, 2 ethylhexyl p-dimethylaminobenzoate; salicylates e.g. 3,3,5-trimethyl-2-hydroxycyclohexyl benzoate (homosalate), 2-ethylhexyl salicylate, triethylamine salicylate; cinnamates e.g. diethanolamine p-methoxycinnamate, 2-ethylhexyl p-methoxicinnamate (parsol MCX®); benzophenones e.g. benzoresorcinol (benzophenone-1), 3,2′,4,4′-tetrahydroxybenzophenone (benzophenone-2), oxybenzone (benzophenone-3), sulisobenzone (benzophenone-4), 2,2′-dihydroxy-4,4′-dimethoxybenzophenone (benzophenone-6), dioxybenzone (benzophenone-8), octabenzone (benzophenone-12); anthranilates e.g. menthil anthranilate; acrylates e.g. 2-ethylhexyl 2-cyano-3,3-diphenyllacrylate (octocrylene), 2-cyano-3,3-diphenyl ethyl acrylate (etocrylene); 1,3 diones e.g. 1-(4-isopropylphenyl)-3-phenylpropano-1,3-dione, 1-(4-tertbutylphenyl)-3-(4-methoxyphenyl)propane-1,3-dione; and others e.g. 2-phenylbenzimidazole-5-sulphonic acid, 3-(4-methylbenzylidene)-d-1-camphor.

A more detailed approach on sunscreens can be found in Nicholas J. Lowe, Nadim A. Shaath & Madhu A. Pathak: Sunscreens Development, Evaluation and Regulatory Aspects (Marcell Dekker, Inc., New York, 1997), in which relative topics are explored concerning to the evolution, photobiologic and regulatory aspects, as well as to the relationships between the chemical structure and the biological activity of products used as sunscreens.

The effectiveness of a sunscreen is described by the sun protection factor (SPF), which is defined as the requested energy dose to produce a minimum erythema (burn) in the protected skin, Tps, divided by the UV energy requested to produce a minimum erythema in the unprotected skin, Tus:

SPF=Tps/Tus

which dose can be measured in light intensity or in time of exposition.

There are two possible ways of obtaining high protection factors:

a) one in which the concentration of the active photoabsorbent is increased, for which, according to the Beer law, as larger the concentration the larger will be number of absorbed photons. However, high concentrations can be irritating to the skin.
b) the other one, the most used to increase SPF, consists of two or more substances combination in the formula (Kimbrough, D. R., J. Chem. Ed., 1997, 74: (1) 51-53).

Researches have shown that the use of sunscreens capable of blocking especifically UV-B rays, but not UV-A, can increase the number of individuals with skin cancer, once it allows people who use it a larger exposition of their skins to the sunshine, for an extended period of time without burns, leading to a 1-2% increase in the maligne types of cancer cases for each 1% reduction in the concentration of ozone. Studies about the UV radiation incidence according to the latitude and the ozone layer depletion, involving inhabitants of many different continents, have shown a significant increase of basal cell, squamous cell carcinomas and cataracts, besides the suppression of humans' immunological systems, resulting in the increase of infectious diseases. It should be emphasized that other biological systems have been altered as the interference in the photosynthetic efficiency of plants, with a smaller production of leaves, flowers and seeds; aquatic food chain imbalance because of the destruction of the phytoplankton that lives close to the surface.

The need of new sunscreens preparation becomes clear when the statistics show an increase in the number of serious problems, caused by the extended exposure to the solar rays. Thus, the present invention seeks, first, to provide new photoprotection substances. However, this invention is not applied just to cosmetic products, but to any composition in which the intention is to protect some object or surface, for instance the skin, inks, plastics, from the ultraviolet rays exposition damages. As it will be demonstrated, the new photoprotection compounds of the present invention are obtained from the cashew nut shell liquid.

The Anarcadiaceae family, to which belongs the cashew tree, involves the Anacardium genus with several different species, where the Anacardium occidentale L. species constitutes the most common variety, native from the Brazilian Northeast and cultivated in many equatorial and sub-equatorial areas of the world (Peixoto, A., 1960, Caju—Produtos Rurais. Ministry of the Agriculture, Service of Agricultural Information, Rio de Janeiro; Johnson, D. V., 1974, The Cashew of the Northeast of Brazil—A Geographical Study. Translation of José Alexandre Robatto Orrico, ETENE/BNB; Alvim Júnior, F., Andrade, M. E., 1985, O caju que um dia foi brasileiro, Ciência Hoje magazine, 3, 67-72; Martinez, M. A.; Barrera, P., 1992 Caju—Uma planta de mil utilidades, Ed. Icon, São Paulo; ETENE/BNB, 1973, Agroindústria do caju no Nordeste—situação atual e perspectivas). The initial interest in the cashew tree cultivation aimed at the pulp processment for industrialization of the juice, rich in sugars, mineral salts, proteins, vitamin C and tannins, without considering the almond, which is rich in proteins and fatty components. Only later, the by-product of the chestnut processment, the liquid of the cashew nut (CNSL), started to be used as raw material in the production of insecticides, germicidal, anti-oxidizers, thermal insulation, attrition material, plasticizers, surfactants, inks, vanishes (Aggarwal, J. S., 1975, Journal of the Colour Society, p. 1-9; Ramaiah, M. S., 1976, Fette-Seifen-Anstrichmittel, 78, 472-477; Attanasi, O., Mountain-Zanetti, F., Perdomi, F., Scagliarini, A., 1979, La Chimica & L'Industria, 61, 718-725). Recently, CNSL has been used as additive for fuels and lubricants (FUNCAP researches. Revista de Ciência e Tecnologia, Fortaleza, September, number 2, pp 14-18, 1999). CNSL is a viscous, dark brown viscous, acrid, caustic and inflammable oil found in the mesocarp alveoli of the cashew chestnut, comprising 25% of the fruit weight, in natura, being considered one of the richest sources of phenolyc lipids: anacardic acids, cardols, cardanols and methylcardols. Only six countries (Brazil, India, Madagascar, Mozambique, Kenya and Tanzania) stand out in a significant way in the production and commercial exploration of the cashew nut. The unsaturated fraction is a compound mixture with one, two or three non-conjugated insaturation, of cis configuration, that are located in the carbons 8′, 11′ and 14′, respectively. The industrial importance of CNSL can be evaluated by the existence of hundreds of international patents and published works, involving the characterization and application of this raw material, that has, recently, been used as source for researches in Brazil (Santos, M. L. & Magalhães, G. C. of, 1999, J. Braz. Chem. Soc., 10, 13-20; Santos, M. L. dos, 1997, Contribuição aproveitamento de matérias-primas abundantes no país em síntese orgânica—Síntese da Lasiodiplodina a partir do LCC. Doctoral dissertation, UnB, 1997; Silva, G. R. dos, Santos, M. L. dos, Pilgrim, L. A. S. & Resck, I. S., 2000, 23a Meeting of the Chemistry Brazilian Society, Abstracts SBQ-QO. 105).

From the chemical point of view, CNSL is configured as a versatile raw material to a series of chemical transformations, due to the phenolic and lipidic constituents' dualistic nature, including the aromatic and acyclic character, associated to the existence of many functional groups in the aromatic ring and presence of multiple unsaturations in the acyclic chain. The CNSL constituents chemical nature, concerning the easiness of obtention and some chemical transformations control in the structure of some of their phenolic constituents described in the literature, led to the elaboration of this proposal that aims at its potential exploration as raw material in the synthesis of new protecting agents against the solar radiation.

### SUMMARY OF THE INVENTION

It is an object of the present invention to provide alternatives for the available photoprotection molecules.

It is another object of this invention to provide the use of molecules derivatives originating from the Anacardium genus species, as photoprotector molecules to give protection against UVA and/or UVB wavelength ultraviolet rays. More specifically, UVA and UVB rays, simultaneously, can be absorbed by some molecules of the present invention.

It is an additional object of the present invention to provide new compositions containing such molecules and their use as a mean of protecting the object or surface in which the exposition to UV radiations is harmful.

It is still another object of the present invention to provide chemical processes for the production of the analyzed molecules.

### BRIEF DESCRIPTION OF THE DRAWINGS

FIG. 1 shows the non-phototoxic answer for V36 in the in vitro phototoxicity test.

FIG. 2 shows the non-phototoxic answer for 8-methoxypsoralen in the in vitro phototoxicity test.

FIG. 3 shows the in vivo phototoxicity test in: answer in guinea pigs—substance V32 application on the left side, 8-methoxypsoralen application on the right side; and the back inferior irradiated area.

FIG. 4 shows the non-irritating answer in albino rabbit for the V33 substance.

### DETAILED DESCRIPTION OF THE INVENTION

After a brief reference to this invention objects, we will, now, describe them in details, using, whenever opportune, the preferential materializations of the invention.

This invention has as one of the innovative characteristics the synthesis of photoprotector agents corresponding to the formulas (I), (II) and (III). These are CNSL-derived molecules rationally planned as sunscreens. These derivatives present as main structural characteristics the photoabsorbents chromophoric patterns found in aromatic, cinnamic, sulphonic esters, as well as conjugated arylketones, necessary to the photoprotector activity, joined to the natural hydrophobic subunit, recognized by the alkilic chain of the CNSL phenolic derivates.

The use of this structural pattern for sunscreens has not been previously reported, and, therefore, the compounds described in this invention and their synthetic methodology represents a change among the organic photoprotector agents.

Additionally, the present invention compounds conjugate, in a single structure, different photoabsorbent chromophores, providing relevant synthesis cost reduction in relation to the isolated molecules found in the literature and in the market.

The new compounds about which this invention is concerned to belong to the phenolic derivative class of the Cashew Nut Shell Liquid e.g. anacardic acids, cardanols, cardols, methylcardols, their homologous and isosteres, of general structure (I):

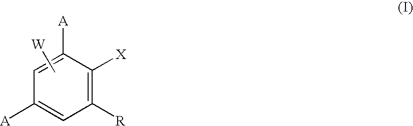

Where R is alkyl, alkenil, octyl, pentadecyl, 1-[(E)-1-pentadecenyl, 1-[(Z)-8-pentadecenyl, 1-[(8Z,11Z)-8,11-pentadecadienyl, 1-[(8Z,11Z)-8,11,14-pentadecatrienyl, cycloalkyl, alkoxyl, B-alkoxyl, B-sulfanyl, B-sulfonyl, B-sulfinyl, B-sulfonates, B-sulfonamides, B-amino, B-carbamoyl, B-halides, B-carboalkoxyl, B-carbothioalkoxyl, N,N-B-carbamoyl, B-trihaloalkane, B-ciano, nitro-B, azido-B, B-amines, B-amides, halides, carboalkoxyl, carbothioalkoxyl, N,N-dissubstituted-carbamoyl, trihaloalkane, ciano, nitro ou azido.
B is hydrogen, alkyl, alkenyl, cicloalkyl, cicloalkenyl or aryl;
X is hydrogen, carboxyl, alkylcarboxyl, alkenylcarboxyl, alkylcarboxylate, alkenylcarboxylate, carbothioate, carbodithioate, carboalkoxyl, carbamoyl, formyl, alkylcarbonyl, arylcarbonyl, (E)-2-propenoic acid, (2E,4E)-2,4-pentadienoic acid, sulfonic acid, (E)-1-ethene-1-sulphonic, (1E,3E)-1,3-butadiene-1-sulfonic acid and its homo-derivated or its alkylic, phenolic, benzylic or cinnamic esters, lactones, amides, lactames and imides, W-benzoyl;
A is hydrogen or R₁
R₁ is hydrogen, hydroxyl, alkyl, cycloalkyl; phenyl, furyl, thiophenyl, pyridinyl, pyrimidinyl, pyrrolyl, thiazolyl, W-quinazolyl, W-isoquinolyl, W-benzimidazolyl, W-benzoxazolyl, W-benzothiazolyl, acyl, acetyl, W-cinnamoyl, chrotyl, W-benzoyl, alkoxyl, cycloalkoxyl, B-alkoxyl, B-sulfanyl, B-sulfonyl, B-sulfinyl, B-sulfonates, B-sulfonamides, B-amino, B-carbamoyl, B-halides, B-carboalkoxyl, B-carbothioalkoxyl, N,N-B-carbamoyl, B-trihaloalkane, B-ciano, nitro-B, azido-B, N,N-di-B-carbamoyl, trihaloalkane;
B is hydrogen, alkyl, alkenyl, cicloalkyl, cicloalkenyl or aryl; and
W is hydrogen, ortho-hydroxyl, ortho-alkyl, ortho-cycloalkyl, ortho-alkoxyl, ortho-cycloalkoxyl, ortho-sulfanyl, ortho-aryloxyl, ortho-sulfones, ortho-sulfides, ortho-sulfinyl, ortho-sulfonates, ortho-sulfonamides, ortho-amine, ortho-amide, ortho-halides, ortho-carboalkoxyl, ortho-carbothioalkoxyl, ortho-carbamoyl, ortho-trihaloalkane, ortho-ciano, ortho-nitro, ortho-acyl, ortho-acetyl, ortho-benzoyl, ortho-4-alkyloxybenzoyl, ortho-4-alkoxybenzoyl ortho-4-methoxybenzoyl, ortho-4-dimethylaminobenzoyl, ortho-cinnamoyl, ortho-4-alkyloxycinnamoyl, ortho-4-methoxycinnamoyl, ortho-3-(4-methoxyphenyl)-3-oxo-propanoyl, ortho-3-(4-alkoxyphenyl)-3-oxo-propanoyl, ortho-3-(4-phenoxyphenyl)-3-oxo-propanoyl, ortho-3-(4-aminophenyl)-3-oxo-propanoyl, ortho-3-(4-carbamoylphenyl)-3-oxo-propanoyl, ortho-3-(4-methoxyphenyl)-1,3-propanodione, ortho-3-(4-alkoxyphenyl)-1,3-propanodione, ortho-3-(4-phenoxyphenyl)-1,3-propanodione, ortho-3-(4-aminophenyl)-1,3-propanodione, ortho-3-(4-carbamoylphenyl)-1,3-propanodione, ortho-2H-benzo[d][1,2,3]triazol-2-yl, meta-hydroxyl, meta-alkyl, meta-cycloalkyl, meta-alkoxyl, meta-cycloalkoxyl, meta-sulfanyl, meta-aryloxyl, meta-sulfones, meta-sulfides, meta-sulfinyl, meta-sulfonates, meta-sulfonamides, meta-amine, meta-amide, meta-halides, meta-carboalkoxyl, meta-carbothioalkoxyl, meta-carbamoyl, meta-trihaloalkane, meta-ciano, meta-nitro, meta-acyl, meta-acetyl, meta-benzoyl, meta-4-alkyloxybenzoyl, meta-4-alkoxybenzoyl, meta-4-methoxybenzoyl, meta-4-dimethyilaminobenzoyl, meta-cinnamoyl, meta-4-alkyloxycinnamoyl, meta-4-methoxycinnamoyl, meta-3-(4-methoxyphenyl)-3-oxo-propanoyl, meta-3-(4-alkoxyphenyl)-3-oxo-propanoyl, meta-3-(4-phenoxyphenyl)-3-oxo-propanoyl, meta-3-(4-aminophenyl)-3-oxo-propanoyl, meta-3-(4-carbamoylphenyl)-3-oxo-propanoyl, meta-3-(4-methoxyphenyl)-1,3-propanodione, meta-3-(4-alkoxyphenyl)-1,3-propanodione, meta-3-(4-phenoxyphenyl)-1,3-propanodione, meta-3-(4-aminophenyl)-1,3-propanodione, meta-3-(4-carbamoylphenyl)-1,3-propanodione, meta-2H-benzo[d][1,2,3]triazol-2-yl, para-hydroxyl, para-alkyl, para-cycloalkyl, para-alkoxyl, para-cycloalkoxyl, para-sulfanyl, para-aryloxyl, para-sulfones, para-sulfides, para-sulfinyl, para-sulfonates, para-sulfonamides, para-amine, para-amide, para-halides, para-carboalkoxyl, para-carbothioalkoxyl, para-carbamoyl, para-trihaloalkane, para-ciano, para-nitro, para-acyl, para-acetyl, para-benzoyl, para-4-alkyloxybenzoyl, para-4-alkoxybenzoyl, para-4-methoxybenzoyl, para-4-dimethyilaminobenzoyl, para-cinnamoyl, para-alkyloxycinnamoyl or para-4-methoxycinnamoyl, para-3-(4-methoxyphenyl)-3-oxo-propanoyl, para-3-(4-alkoxyphenyl)-3-oxo-propanoyl, para-3-(4-phenoxyphenyl)-3-oxo-propanoyl, para-3-(4-aminophenyl)-3-oxo-propanoyl, para-3-(4-carbamoylphenyl)-3-oxo-propanoyl, para-3-(4-methoxyphenyl)-1,3-propanodione, para-3-(4-alkoxyphenyl)-1,3-propanodione, para-3-(4-phenoxyphenyl)-1,3-propanodione, para-3-(4-aminophenyl)-1,3-propanodione, para-3-(4-carbamoylphenyl)-1,3-propanodione, para-2H-benzo[d][1,2,3]triazol-2-yl.

This invention compounds also belong to the class of the phenolic derivative class of the Cashew Nut Shell Liquid e.g. anacardic acids, cardanols, cardols, methylcardols, their homologous and isosteres, of general structure (II)

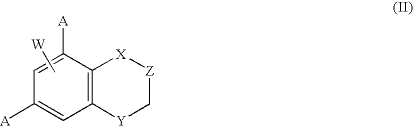

where Y is C₁-C₈ alkyl optionally substituted with one carbonyl, hydroxyl, thiol, halide or amine; C₁-C₈ alkenyl optionally substituted with a carbonyl, hydroxyl, thiol, halide or amine; 8-(1-octanol), 8-(E)-7-octen-1-ol, 8-(E)-6-ceto-7-octen-1-ol, 8-(1-octanethiol), 8-(E)-7-octene-1-thiol, 8-(E)-6-ceto-7-octene-1-thiol, 8-(1-octanamine), 8-(E)-7-octen-1-amine, 8-(E)-6-ceto-7-octen-1-amine;
Z is oxygen, sulfur, methylene, carbonyl, thiocarbonyl, sulfinyl, sulfonyl or azo;
X is carbonyl, thiocarbonyl, sulfanyl, sulfinyl, sulfonyl, hydroxyl, sulfanyl, methylene or azo;
A is hydrogen or R₁
R₁ is hydrogen, hydroxyl, alkyl, cycloalkyl; phenyl, furyl, thiophenyl, pyridinyl, pyrimidinyl, pyrrolyl, thiazolyl, W-quinazolyl, W-isoquinolyl, W-benzimidazolyl, W-benzoxazolyl, W-benzothiazolyl, acyl, acetyl, W-cinnamoyl, chrotyl, W-benzoyl, alkoxyl, cycloalkoxyl, B-alkoxyl, B-sulfanyl, B-sulfonyl, B-sulfinyl, B-sulfonates, B-sulfonamides, B-amino, B-carbamoyl, B-halides, B-carboalkoxyl, B-carbothioalkoxyl, N,N-B-carbamoyl, B-trihaloalkane, B-ciano, nitro-B, azido-B, N,N-di-B-carbamoyl, trihaloalkane;
B is hydrogen, alkyl, alkenyl, cicloalkyl, cicloalkenyl or aryl; and
W is hydrogen, ortho-hydroxyl, ortho-alkyl, ortho-cycloalkyl, ortho-alkoxyl, ortho-cycloalkoxyl, ortho-sulfanyl, ortho-aryloxyl, ortho-sulfones, ortho-sulfides, ortho-sulfinyl, ortho-sulfonates, ortho-sulfonamides, ortho-amine, ortho-amide, ortho-halides, ortho-carboalkoxyl, ortho-carbothioalkoxyl, ortho-carbamoyl, ortho-trihaloalkane, ortho-ciano, ortho-nitro, ortho-acyl, ortho-acetyl, ortho-benzoyl, ortho-4-alkyloxybenzoyl, ortho-4-alkoxybenzoyl ortho-4-methoxybenzoyl, ortho-4-dimethylaminobenzoyl, ortho-cinnamoyl, ortho-4-alkyloxycinnamoyl, ortho-4-methoxycinnamoyl, ortho-3-(4-methoxyphenyl)-3-oxo-propanoyl, ortho-3-(4-alkoxyphenyl)-3-oxo-propanoyl, ortho-3-(4-phenoxyphenyl)-3-oxo-propanoyl, ortho-3-(4-aminophenyl)-3-oxo-propanoyl, ortho-3-(4-carbamoylphenyl)-3-oxo-propanoyl, ortho-3-(4-methoxyphenyl)-1,3-propanodione, ortho-3-(4-alkoxyphenyl)-1,3-propanodione, ortho-3-(4-phenoxyphenyl)-1,3-propanodione, ortho-3-(4-aminophenyl)-1,3-propanodione, ortho-3-(4-carbamoylphenyl)-1,3-propanodione, ortho-2H-benzo[d][1,2,3]triazol-2-yl, meta-hydroxyl, meta-alkyl, meta-cycloalkyl, meta-alkoxyl, meta-cycloalkoxyl, meta-sulfanyl, meta-aryloxyl, meta-sulfones, meta-sulfides, meta-sulfinyl, meta-sulfonates, meta-sulfonamides, meta-amine, meta-amide, meta-halides, meta-carboalkoxyl, meta-carbothioalkoxyl, meta-carbamoyl, meta-trihaloalkane, meta-ciano, meta-nitro, meta-acyl, meta-acetyl, meta-benzoyl, meta-4-alkyloxybenzoyl, meta-4-alkoxybenzoyl, meta-4-methoxybenzoyl, meta-4-dimethyilaminobenzoyl, meta-cinnamoyl, meta-4-alkyloxycinnamoyl, meta-4-methoxycinnamoyl, meta-3-(4-methoxyphenyl)-3-oxo-propanoyl, meta-3-(4-alkoxyphenyl)-3-oxo-propanoyl, meta-3-(4-phenoxyphenyl)-3-oxo-propanoyl, meta-3-(4-aminophenyl)-3-oxo-propanoyl, meta-3-(4-carbamoylphenyl)-3-oxo-propanoyl, meta-3-(4-methoxyphenyl)-1,3-propanodione, meta-3-(4-alkoxyphenyl)-1,3-propanodione, meta-3-(4-phenoxyphenyl)-1,3-propanodione, meta-3-(4-aminophenyl)-1,3-propanodione, meta-3-(4-carbamoylphenyl)-1,3-propanodione, meta-2H-benzo[d][1,2,3]triazol-2-yl, para-hydroxyl, para-alkyl, para-cycloalkyl, para-alkoxyl, para-cycloalkoxyl, para-sulfanyl, para-aryloxyl, para-sulfones, para-sulfides, para-sulfinyl, para-sulfonates, para-sulfonamides, para-amine, para-amide, para-halides, para-carboalkoxyl, para-carbothioalkoxyl, para-carbamoyl, para-trihaloalkane, para-ciano, para-nitro, para-acyl, para-acetyl, para-benzoyl, para-4-alkyloxybenzoyl, para-4-alkoxybenzoyl, para-4-methoxybenzoyl, para-4-dimethyilaminobenzoyl, para-cinnamoyl, para-alkyloxycinnamoyl or para-4-methoxycinnamoyl, para-3-(4-methoxyphenyl)-3-oxo-propanoyl, para-3-(4-alkoxyphenyl)-3-oxo-propanoyl, para-3-(4-phenoxyphenyl)-3-oxo-propanoyl, para-3-(4-aminophenyl)-3-oxo-propanoyl, para-3-(4-carbamoylphenyl)-3-oxo-propanoyl, para-3-(4-methoxyphenyl)-1,3-propanodione, para-3-(4-alkoxyphenyl)-1,3-propanodione, para-3-(4-phenoxyphenyl)-1,3-propanodione, para-3-(4-aminophenyl)-1,3-propanodione, para-3-(4-carbamoylphenyl)-1,3-propanodione, para-2H-benzo[d][1,2,3]triazol-2-yl.

This invention compounds also belong to the class of the phenolic derivative class of the Cashew Nut Shell Liquid e.g. anacardic acids, cardanols, cardols, methylcardols, their homologous and isosteres, of general structure (III)

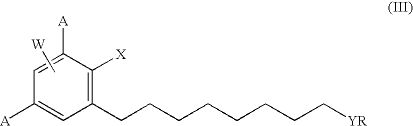

where X is hydrogen, carboxyl, alkylcarboxyl, alkenylcarboxyl, alkylcarboxylate, alkenylcarboxylate, carbothioate, carbodithioate, carboalkoxyl, carbamoyl, formyl, alkylcarbonyl, arylcarbonyl, (E)-2-propenoic acid, (2E,4E)-2,4-pentadienoic acid, sulfonic acid, (E)-1-ethene-1-sulphonic, (1E,3E)-1,3-butadiene-1-sulfonic acid and its homo-derivated or its alkylic, phenolic, benzylic or cinnamic esters, lactones, amides, lactames and imides, W-benzoyl;
Y is oxygen, sulfur, methylene, carbonyl, thiocarbonyl, carboxyl, carbothioate, carbodithioate, carboalkoxyl, carbamoyl, sulfinyl, sulfonyl or azo;
A is hydrogen or R₁
R and R₁ are, independently, hydrogen, hydroxyl, alkyl, alkenyl, alkinyl, cycloalkyl, cycloalkoxyl, B-alkoxyl, B-sulfanyl, B-sulfonyl, B-sulfinyl, B-sulfonates, B-sulfonamides, B-amino, B-carbamoyl, B-halides, B-carboalkoxyl, B-carbothioalkoxyl, N,N-B-carbamoyl, B-trihaloalkane, B-ciano, nitro-B, azido-B, alkoxyl, phenyl, furyl, thiophenyl, pyridinyl, pyrimidinyl, pyrrolyl, thiazolyl, W-quinazolyl, W-isoquinolyl, W-benzimidazolyl, W-benzoxazolyl, W-benzothiazolyl, acyl, acetyl, W-cinnamoyl, chrotyl, W-benzoyl, N,N-di-B-carbamoyl, trihaloalkane;
B is hydrogen, alkyl, alkenyl, cicloalkyl, cicloalkenyl or aryl; and
W is hydrogen, ortho-hydroxyl, ortho-alkyl, ortho-cycloalkyl, ortho-alkoxyl, ortho-cycloalkoxyl, ortho-sulfanyl, ortho-aryloxyl, ortho-sulfones, ortho-sulfides, ortho-sulfinyl, ortho-sulfonates, ortho-sulfonamides, ortho-amine, ortho-amide, ortho-halides, ortho-carboalkoxyl, ortho-carbothioalkoxyl, ortho-carbamoyl, ortho-trihaloalkane, ortho-ciano, ortho-nitro, ortho-acyl, ortho-acetyl, ortho-benzoyl, ortho-4-alkyloxybenzoyl, ortho-4-alkoxybenzoyl ortho-4-methoxybenzoyl, ortho-4-dimethylaminobenzoyl, ortho-cinnamoyl, ortho-4-alkyloxycinnamoyl, ortho-4-methoxycinnamoyl, ortho-3-(4-methoxyphenyl)-3-oxo-propanoyl, ortho-3-(4-alkoxyphenyl)-3-oxo-propanoyl, ortho-3-(4-phenoxyphenyl)-3-oxo-propanoyl, ortho-3-(4-aminophenyl)-3-oxo-propanoyl, ortho-3-(4-carbamoylphenyl)-3-oxo-propanoyl, ortho-3-(4-methoxyphenyl)-1,3-propanodione, ortho-3-(4-alkoxyphenyl)-1,3-propanodione, ortho-3-(4-phenoxyphenyl)-1,3-propanodione, ortho-3-(4-aminophenyl)-1,3-propanodione, ortho-3-(4-carbamoylphenyl)-1,3-propanodione, ortho-2H-benzo[d][1,2,3]triazol-2-yl, meta-hydroxyl, meta-alkyl, meta-cycloalkyl, meta-alkoxyl, meta-cycloalkoxyl, meta-sulfanyl, meta-aryloxyl, meta-sulfones, meta-sulfides, meta-sulfinyl, meta-sulfonates, meta-sulfonamides, meta-amine, meta-amide, meta-halides, meta-carboalkoxyl, meta-carbothioalkoxyl, meta-carbamoyl, meta-trihaloalkane, meta-ciano, meta-nitro, meta-acyl, meta-acetyl, meta-benzoyl, meta-4-alkyloxybenzoyl, meta-4-alkoxybenzoyl, meta-4-methoxybenzoyl, meta-4-dimethyilaminobenzoyl, meta-cinnamoyl, meta-4-alkyloxycinnamoyl, meta-4-methoxycinnamoyl, meta-3-(4-methoxyphenyl)-3-oxo-propanoyl, meta-3-(4-alkoxyphenyl)-3-oxo-propanoyl, meta-3-(4-phenoxyphenyl)-3-oxo-propanoyl, meta-3-(4-aminophenyl)-3-oxo-propanoyl, meta-3-(4-carbamoylphenyl)-3-oxo-propanoyl, meta-3-(4-methoxyphenyl)-1,3-propanodione, meta-3-(4-alkoxyphenyl)-1,3-propanodione, meta-3-(4-phenoxyphenyl)-1,3-propanodione, meta-3-(4-aminophenyl)-1,3-propanodione, meta-3-(4-carbamoylphenyl)-1,3-propanodione, meta-2H-benzo[d][1,2,3]triazol-2-yl, para-hydroxyl, para-alkyl, para-cycloalkyl, para-alkoxyl, para-cycloalkoxyl, para-sulfanyl, para-aryloxyl, para-sulfones, para-sulfides, para-sulfinyl, para-sulfonates, para-sulfonamides, para-amine, para-amide, para-halides, para-carboalkoxyl, para-carbothioalkoxyl, para-carbamoyl, para-trihaloalkane, para-ciano, para-nitro, para-acyl, para-acetyl, para-benzoyl, para-4-alkyloxybenzoyl, para-4-alkoxybenzoyl, para-4-methoxybenzoyl, para-4-dimethyilaminobenzoyl, para-cinnamoyl, para-alkyloxycinnamoyl or para-4-methoxycinnamoyl, para-3-(4-methoxyphenyl)-3-oxo-propanoyl, para-3-(4-alkoxyphenyl)-3-oxo-propanoyl, para-3-(4-phenoxyphenyl)-3-oxo-propanoyl, para-3-(4-aminophenyl)-3-oxo-propanoyl, para-3-(4-carbamoylphenyl)-3-oxo-propanoyl, para-3-(4-methoxyphenyl)-1,3-propanodione, para-3-(4-alkoxyphenyl)-1,3-propanodione, para-3-(4-phenoxyphenyl)-1,3-propanodione, para-3-(4-aminophenyl)-1,3-propanodione, para-3-(4-carbamoylphenyl)-1,3-propanodione, para-2H-benzo[d][1,2,3]triazol-2-yl.

Formula (I), (II) and (III) compounds were obtained in a good to excellent yields, using the described synthetic methodology. This synthesis methodology is characterized by presenting few stages, with high yields and using as a starting point commercially available compounds, what qualifies this synthetic methodology for industrial use.

The present invention compounds were planned through convergent synthesis, making use of classical reactions such as:
O-alkylation
O-esterification/Lactonization;
C-acylation via enolates
FISCHER esterification
KNOEVENAGEL condensation
DOEBNER condensation
Ozonolysis;
Catalytic hydrogenation with Pd/C;
Oxidation;
Reduction with metallic hydrides.
FRIEDEL-CRAFTS acylation;
FRIES rearrangement;
BAKER-VENKATARAMAN rearrangement
GRIGNARD reaction;
DAKIN reaction;
ELBS persulfate oxidation;
Formylation;
Sulfonation;

More specifically, formula (I) compounds of the present invention can be prepared through a process comprising the following steps:
Phenolic hydroxyl esterification or etherification of the saturated and unsaturated cardols;
Catalytic hydrogenation with Pd/C;
Formylation with zinc cyanide [ZN(CN₂)] in THF/ethylic ether with gaseous HCl bubbling (J. Braz. Chem. Soc. 10 (1): 13-20, 1999);
Selective oxidation to the corresponding acid with sodium chlorite;
FISCHER esterification;
DOEBNER condensation of the aldehyde with malonic acid as well as its derivative esters in pyridine catalyzed by piperidine;
FRIES rearrangement of O-acylates derivatives e.g. O-benzoates and O-cinnamates W-substituted catalyzed by Lewis acids e.g. anhydrous aluminum chloride;
BAKER-VENKATARAMAN rearrangement of O-acylates derivatives e.g. O-benzoates and O-cinnamates W-substituted catalyzed by bases e.g. sodium hydroxide;
C-acylation reaction, through enolates, of acetophenonic derivatives benzoyl halides and cinnamoyl W-substituted catalyzed by bases e.g. sodium hydroxide;
FRIEDEL-CRAFTS reaction of the O-acylates e.g. O-benzoates and O-cinnamates W-substituted catalyzed by Lewis acids e.g. anhydrous aluminum chloride;
GRIGNARD reaction with mixed acids halides and phenylmagnesium halides;
DAKIN reaction with hydrogen peroxide or peracids of formyl derivatives;
ELBS persulfate oxidation of phenolic derivatives e.g. cardol, cardanol and anacardic acids;

More specifically, formula (II) compounds of the present invention can be prepared through a process comprising the following steps:
Phenolic hydroxyl esterification or etherification of the CNSL saturated and unsaturated cardols;
Ozonolysis;
Reduction with NaBH₄;
GATTERMANN reaction—Formylation with zinc cyanide [ZN(CN₂)] in THF/ethylic ether with gaseous HCl bubbling (J. Braz. Chem Soc. 10 (1): 13-20, 1999);
Selective oxidation to the corresponding acid with sodium chlorite;
FISCHER esterification;
DOEBNER condensation of the aldehyde with malonic acid as well as its derivative esters in pyridine catalyzed by piperidine;
FRIES rearrangement of O-acylates derivatives e.g. O-benzoates and O-cinnamates W-substituted catalyzed by Lewis acids e.g. anhydrous aluminum chloride;
BAKER-VENKATARAMAN rearrangement of O-acylates derivatives e.g. O-benzoates and O-cinnamates W-substituted catalyzed by bases e.g. sodium hydroxide;
C-acylation reaction, through enolates, of acetophenonic derivatives benzoyl halides and cinnamoyl W-substituted catalyzed by bases e.g. sodium hydroxide;
FRIEDEL-CRAFTS reaction of the O-acylates e.g. O-benzoates and O-cinnamates W-substituted catalyzed by Lewis acids e.g. anhydrous aluminum chloride;
GRIGNARD reaction with mixed acids halides and phenylmagnesium halides;
DAKIN reaction with hydrogen peroxide or peracids of formyl derivatives;
ELBS persulfate oxidation of phenolic derivatives e.g. cardol, cardanol and anacardic acids;
Catalyzed lactonization through 2-chlorine-1-methylpyridine iodide

More specifically, formula (III) compounds of the present invention can be prepared through a process comprising the following steps:
Phenolic hydroxyl esterification or etherification of the CNSL saturated and unsaturated cardols;
Ozonolysis;
Reduction with NaBH₄;
GATTERMANN reaction—Formylation with zinc cyanide [ZN(CN₂)] in THF/ethylic ether with gaseous HCl bubbling (J. Braz. Chem. Soc. 10 (1): 13-20, 1999);
Selective oxidation to the corresponding acid with sodium chlorite;
FISCHER esterification;
DOEBNER condensation of the aldehyde with malonic acid as well as its derivative esters in pyridine catalyzed by piperidine;
FRIES rearrangement of O-acylates derivatives e.g. O-benzoates and O-cinnamates W-substituted catalyzed by Lewis acids e.g. anhydrous aluminum chloride;
BAKER-VENKATARAMAN rearrangement of O-acylates derivatives e.g. O-benzoates and O-cinnamates W-substituted catalyzed by bases e.g. sodium hydroxide;
C-acylation reaction, through enolates, of acetophenonic derivatives benzoyl halides and cinnamoyl W-substituted catalyzed by bases e.g. sodium hydroxide;
FRIEDEL-CRAFTS reaction of the O-acylates e.g. O-benzoates and O-cinnamates W-substituted catalyzed by Lewis acids e.g. anhydrous aluminum chloride;
GRIGNARD reaction with mixed acids halides and phenylmagnesium halides;
DAKIN reaction with hydrogen peroxide or peracids of formyl derivatives;
ELBS persulfate oxidation of phenolic derivatives e.g. cardol, cardanol and anacardic acids;
Esterification of the primary and secondary alcohols or thiols from the lateral chain with mixed anhydride or acids cloride.
Amidation of the primary or secondary amines from the lateral chain with mixed anhydride or acids chloride

The processes above mentioned don't limit the invention; they are useful just as examples of one of the countless ways of carrying it out.

In this report, to illustrate, we describe the compound synthesis:
2-hydroxy-6-pentadecylbenzoic acid
2-hydroxy-6-[(E)-1-pentadecenyl]benzoic acid
2-hydroxy-6-[(Z)-8-pentadecenyl]benzoic acid
2-hydroxy-6-[(8Z,11Z)-8,11-pentadecadienyl]benzoic acid
2-hydroxy-6-[(8Z,11Z)-8,11,14-pentadecatrienyl]benzoic acid
2-methylcarbonyloxy-6-pentadecylbenzoic acid
2-methylcarbonyloxy-6-[(E)-1-pentadecenyl]benzoic acid
2-methylcarbonyloxy-6-[(Z)-8-pentadecenyl]benzoic acid
2-methylcarbonyloxy-6-[(8Z,11Z)-8,11-pentadecadienyl]benzoic acid
2-methylcarbonyloxy-6-[(8Z,11Z)-8,11,14-pentadecatrienyl]benzoic acid
Methyl 2-methoxy-6-pentadecylbenzoate
Methyl 2-methoxy-6-[(E)-1-pentadecenyl]benzoate
Methyl 2-methoxy-6-[(Z)-8-pentadecenyl]benzoate
Methyl 2-methoxy-6-[(8Z,11Z)-8,11-pentadecadienyl]benzoate
Methyl 2-methoxy-6-[(8Z,11Z)-8,11,14-pentadecatrienyl]benzoate
3-pentadecylphenol
3-[(E)-1-pentadecenyl}phenol
3-[(Z)-8-pentadecenyl]phenol
3-[(8Z,11Z)-8,11-pentadecadienyl]phenol
3-[(8Z,11Z)-8,11,14-pentadecatrienyl]phenol
3-pentadecenylphenyl acetate
3-pentadecenylphenyl acrylate
3-pentadecyl-1-phenyl carbonyloxy benzene
1-methoxy-3-pentadecylbenzene
2-hydroxy-4-pentadecylphenyl-phenylmetanone
4-hydroxy-2-pentadecylphenyl-phenylmetanone
2-methoxy-4-pentadecylphenyl-phenylmetanone
4-methoxy-2-pentadecylphenyl-phenylmetanone
2-hydroxy-6-pentadecylphenyl-phenylmetanone
2-methoxy-6-pentadecylphenyl-phenylmetanone
3-methoxy-1-(8-phenylcarbonyloxy octyl)benzeno
3-methoxy-1-[(8-(4-methoxy phenyl carboniloxy)octyl]benzeno
3-phenyl-(E)-2-propenoate of 8<3-methoxyphenyl)octyl
3-(4-methoxyphenyl)-(E)-2-propenoate of 8-(3-methoxyphenyl)octyl
1-phenylcarbonyloxy-3-(8-phenylcarbonyloxyoctyl)benzene
1-(4-methoxyphenylcarbonyloxy)-3-[8-(4-methoxyphenylcarbonyloxyoctyl]benzeno
3-phenyl-(E)-2-propenoate of 3-{8-[2-phenyl-(E)-1-ethenylcarbonyloxy]octyl}phenyl
3-(4-methoxyphenyl)-(E)-2-propenoate of 3-{8-[2-(4-methoxyphenyl-(E)-1-ethenylcarbonyloxy]octyl}phenyl

A detailed description of the synthetic methods of this invention for some of the demanded compounds is next explained, and the relevant spectroscopic data is included to its characterization. Additionally, mutagenicity and genotoxicity tests, among others, are described, showing that the present invention compounds are adequaded for the respective use. The following examples illustrate, but they don't limit to present invention.

### EXAMPLE 1

### Obtantion of Unsaturated Anacardic Acids from CNSL

### General Procedure

It was obtained 20.0 g of CNSL from 453 g of cashew nut shell, through the expression process (cold compress). The shells were separate from the chestnuts, cut in small pieces and grinded with a home-made grinder, to separate the liquid. The unsaturated anacardic acids (MM=344.18 g mol⁻¹) were extracted, as anacardate, of raw CNSL through Pb(OH)₂ treatment.

In an Erlenmeyer 125 mL, 4.6 g of Pb(NO₃)₂ were solubilized in 17.5 mL of distilled water and, under constant agitation, 1.2 g of NaOH solubilized in 7.0 mL of water were added to the solution. After 1 hour, the suspension was filtered under vacuum and the precipitate (Pb(OH)₂) was washed with water until the filtrate has reached a neutral pH and, finally, it was washed with ethanol (10.0 mL). The obtained precipitate was transferred for an Erlenmeyer and 2.6 g of natural CNSL solubilized in ethanol (17.0 mL) were added. The mixture was under constant agitation for 2 hours, when then the precipitate was collected with a vacuum filtration and washed with ethanol. The collected filtrate, constituted of cardanol, cardol and 2-methylcardol, was stored for subsequent treatment. The obtained precipitate, consisting of lead anacardates, was suspended in 20.0 mL of ethyl ether and 10.0 mL of a HNO₃ 20% solution. After 1 hour under constant agitation, the suspension was vacuum filtered and the collected liquid filtrate was transferred to a separation funnel, where the organic phase was washed with water (40 mL) until that the aqueous phase got pH˜6 and it was brine washed twice (2×40 mL). Finally, the organic phase was dried under anhydrous Na₂SO₄, filtered under active celite/coal and the solvent vacuum evaporated, to originate a dark oil (unsaturated anacardic acids) with 59% yield in relation to the total mass and 97% in relation to 60% of anacardic acids present in natural CNSL.

IR (film) υₘₐₓ: 3584-2547, 3009, 2925, 2854, 1646, 1608, 1448, 1246, 1211 cm⁻¹.

¹H NMR (200 MHz, CDCl₃): δ: 0.8-1.0 (m, 3H); 1.0-2.1 (m, n″H); 2.5-2.7 (m, n′H); 2.8-3.0 (m, 2H); 4.6-5.7 (m, nH); 6.4-6.7 (m, 2H); 7.1 (t, J=8.1 Hz, 1H); 10.1 (sl, 2H)

### EXAMPLE 2

### Obtantion of Unsaturated Cardols from CNSL

### General Procedure

The unsaturated cardols (MM=315.62 g mol⁻¹) were separated from the ethanolic filtrate, obtained in the CNSL treatment with Pb(OH)₂, through a chromatographic column eluted with hexane:ethyl acetate 30%, being obtained, after the solvent evaporation, a yield of 23% in relation to the total applied mass and 93% in relation to 24% present in natural CNSL.

IR (film): υₘₐₓ: 3347, 3010, 2926, 2854, 1598, 1465, 1338, 1155 cm⁻¹.

¹H NMR (200 MHz, CDCl₃): δ: 0.8-1.1 (m, 3H); 1.2-2.2 (m, n″H); 2.3-2.6 (m, 2H); 2.7-3.0 (m, n′H); 4.95-6.0 (m, nH); 6.1-6.4 (m, 3H); 6.6-7.5 (sl, 2H).

### EXAMPLE 3

### Obtantion of Unsaturated Cardanols from CNSL

### General Procedure

The unsaturated cardanols (MM=300.19 g mol⁻¹) were obtained as an oil from the extraction of technical CNSL through:

i) chromatographic column eluded with hexane:ethyl acetate 5%, with 62% yield in relation to the total applied mass and a 95% in relation to 65% of present cardanols in technical CNSL;
ii) reduced pressure distillation in the Kugelrohr oven (steam temperature of 180° C.), with a 46% yield in relation to the total mass and a 71% in relation to 65% of present cardanols in technical CNSL.

IR (film): υₘₐₓ: 3363, 3009, 2926, 2854, 1589, 1486, 1456, 1351, 1266 cm⁻¹.

¹H NMR (200 MHz, CDCl₃): δ: 0.7-1.0 (m, 3H); 1.1-2.1 (m, n″H); 2.2-2.4 (m, 2H); 2.5-2.8 (m, n′H); 4.6-5.7 (m, nH); 6.2-6.5 (m, 3H); 6.6-6.8 (m, 1H).

### EXAMPLE 4

### Reactions of Catalytic Hydrogenation of the CNSL Phenolic Derivatives

### General Procedure

In an appropriate flask for the hydrogenation system, 2.0 g of the substratum were solubilized in 20.0 mL of ethanol and, to this solution, 0.106 g with 10% of Pd/C as a catalyzer were added. The mixture was coupled to the hydrogenation system with a 60 psi pressure (˜4 atm), where it was kept for 6 hours, under constant agitation and at room temperature. The mixture was filtered under active celite/coal and the solvent evaporated to originate the saturated solid product, characterized as shown:

Saturated Anacardic Acid—99% Yield

IR (KBr): υₘₐₓ: 3412-2598, 2917, 2850, 1655, 1604, 1466, 1446, 1248 cm⁻¹.

¹H NMR (200 MHz, CDCl₃): δ: 0.8-0.9 (m, 3H); 1.1-1.8 (m, 26H); 2.9-3.1 (m, 2H); 6.8-7.0 (m, 2H); 7.3-7.5 (m, 1H)

Saturated Cardanol—100% Yield

IR (KBr) υₘₐₓ: 3360, 2915, 2848, 1618, 1586, 1499, 1463, 1365, 1264 cm⁻¹.

¹H NMR (200 MHz, CDCl₃): δ: 0.8-0.9 (m, 3H); 1.0-1.7 (m, 26H); 2.4-2.6 (m, 2H); 4.5 (s, 1H); 6.3-6.7 (m, 3H); 6.8-7.1 (m, 1H).

Saturated Cardol—98% Yield

IR (film): υₘₐₓ: 3326, 2916, 2848, 1605, 1509, 1469, 1379, 1201 cm⁻¹.

¹H NMR (200 MHz, CDCl₃): δ: 0.8-0.9 (m, 3H); 1.1-1.7 (m, 26H); 2.3-2.5 (m, 2H); 4.5 (s, 2H); 5.9-6.1 (m, 3H).

### EXAMPLE 5

### Reactions of the CNSL Cardanolic and Cardolic Derivatives Acetylation

### General Procedure

In a flask containing 0.499 g (1.64 mmol) of saturated cardanol solubilized in 4.0 mL of CH₂Cl₂ (0.41 M) under constant agitation, it was added 0.18 mL (1.97 mmol) of acetic anhydride. The solution was cooled in ice bath and it was added, by drops, 0.21 mL (2.46 mmol) of pyridine. After its addition, the reaction mixture was left to reach room temperature, where, under agitation, it stood for 42 hours. The mixture was diluted with CH₂Cl₂ (15 mL) and washed twice with a solution of HCl 1% (2×15 mL) and twice with saturated solution of NaHCO₃ (2×15 mL). The organic phase was dried under anhydrous Na₂SO₄ and the solvent was evaporated, originating acetylated saturated cardanol with a yield of 97%.

For acetylation of the unsaturated cardol 2.0 acetic anhydride equivalent and 2.5 base equivalent were used.

Saturated Cardanol—97% Yield

IR (KBr) υₘₐₓ: 2916, 2849, 1759, 1612, 1587, 1471, 1370, 1206, 1142 cm⁻¹.

¹H NMR (200 MHz, CDCl₃): δ: 0.8-0.9 (m, 3H); 1.1-1.7 (m, 26H); 2.2 (s, 3H); 2.4-2.6 (m, 2H); 6.6-7.1 (m, 4H).

Saturated Cardol—33% Yield (from the Natural CNSL)

IR (film): υₘₐₓ: 2917, 2849, 1774, 1616, 1592, 1470, 1368, 1214, 1190 cm⁻¹.

¹H NMR (200 MHz, CDCl₃): δ: 0.8-1.0 (m, 3H); 1.1-1.9 (m, 26H); 2.3 (s, 6H); 2.5-2.8 (m, 2H); 6.8-7.0 (m, 3H).

Unsaturated Cardanol—67% Yield

IR (KBr) υₘₐₓ: 3009, 2926, 2855, 1769, 1612, 1587, 1487, 1445, 1369, 1206, 1143, 1014 cm⁻¹.

¹H NMR (200 MHz, CDCl₃): δ: 0.7-0.9 (m, 3H); 1.1-2.0 (m, n″H); 2.1 (s, 3H); 2.4-2.6 (m, 2H); 2.6-2.8 (m, n′H); 4.6-5.8 (m, nH); 6.5-7.1 (m, 4H).

Unsaturated Cardol—92% Yield

IR (film): υₘₐₓ: 3010, 2928, 2855, 1772, 1618, 1591, 1451, 1369, 1197, 1123, 1022 cm⁻¹.

¹H NMR (200 MHz, CDCl₃): δ: 0.8-1.0 (m, 3H); 1.2-2.2 (m, n″H); 2.3 (s, 6H); 2.5-2.8 (m, 2H); 2.8-3.1 (m, n′H); 4.9-6.1 (m, nH); 6.7-7.0 (m, 3H).

### EXAMPLE 6

### Reactions of Anacardic Acids Acetylation

### General Procedure

In a flask containing 0.43 mL (4.6 mmol) of acetic anhydride, 2 drops of concentrated H₂SO₄ were added. This solution was under agitation, at room temperature, for 5 minutes, when it was added 0.795 g (2.31 mmol) of unsaturated anacardic acid solubilized in 2.5 mL of acetic anhydride (reaction solvent) and, subsequently, it was heated in oil bath (˜70° C.), for 30 minutes. The reaction was followed by CCD. To the reaction mixture ca of 15 mL of CH₂Cl₂ were added and it was water washed twice with water (2×15 mL), saturated solution of NaHCO₃ (15 mL) until the aqueous phase reached pH˜6.5 and once with brine (15 mL). The organic phase was dried under anhydrous Na₂SO₄ and the solvent was evaporated to originate the acetylated product with 97% yield.

Saturated Anacardic Acid—97% Yield

IR (film): υₘₐₓ: 3419, 2919, 2849, 1775, 1697, 1603, 1576, 1461, 1369, 1290, 1208, 1019 cm⁻¹.

¹H NMR (200 MHz, CDCl₃): δ: 0.8-1.0 (m, 3H); 1.1-1.8 (m, 26H); 2.3 (s, 3H); 2.7-2.9 (m, 2H); 7.0-7.6 (m, 3H); 8.7 (sl, 1H).

Unsaturated Anacardic Acid—75% Yield

IR (KBr) max: 3584-2637, 3009; 2927, 2855, 1773, 1739, 1606, 1577, 1462, 1370, 1199, 1021 cm⁻¹.

¹H NMR (200 MHz, CDCl₃): δ: 0.8-1.0 (m, 3H); 1.1-2.0 (m, n″H); 2.1 (s, 3H); 2.5-2.8 (m, n″H); 4.6-5.8 (m, nH); 6.7-7.1 (m, 2H); 7.1-7.4 (m, 1H); 8.4 (sl, 1H).

### EXAMPLE 7

### Reactions of the Anacardic Acid Methylation

### General Procedure

It was added, in a flask containing 0.485 g (1.41 mmol) of unsaturated anacardic acid, solubilized in 7.0 mL of CH₂Cl₂, 1.92 mL of NaOH 3 M solution, 0.05 mL of the phase transfer catalyst Aliquat® 336 and, last, under constant agitation, 0.92 mL (9.76 mmol) of (CH₃)₂SO₄. The mixture was under agitation, at room temperature, for 30 minutes when, followed by CCD, the end of the reaction was verified. The mixture was diluted with CH₂Cl₂ (20 mL) and washed once with water (20 mL), twice with NH₄OH 2 M solution (2×15 mL) and twice with brine (2×20 mL). The organic phase was dried under anhydrous Na₂SO₄ and the solvent was evaporated to originate the dimethylated product with a 75% yield, purified through chromatographic column.

Saturated Anacardic Acid—75% Yield

IR (film): υₘₐₓ: 3008; 2927, 2854, 1735, 1584, 1470, 1431, 1265, 1111, 1075 cm⁻¹.

¹H NMR (200 MHz, CDCl₃): δ: 0.8-1.0 (m, 3H); 1.1-2.2 (m, n″H); 2.4-2.6 (m, 2H); 3.8 (s, 6H); 5.2-6.0 (m, nH); 6.6-7.0 (m, 2H); 7.2 (t, J=8.1 Hz, 1H).

Unsaturated Anacardic Acid—68% Yield

IR (KBr) υₘₐₓ: 2924, 2853, 1735, 1584, 1470, 1431, 1377, 1267, 1189, 1110, 1075 cm⁻¹.

¹H NMR (200 MHz, CDCl₃): δ: 0.8-1.0 (m, 3H); 1.2-1.7 (m, 26H); 2.5-2.7 (m, 2H); 3.8 (s, 3H); 3.9 (s, 3H); 6.7-7.0 (m, 2H); 7.3 (t, J=8.1 Hz, 1H).

### EXAMPLE 8

### Reactions of the Saturated Cardanol Esterification

### General Procedure

In a flask containing 0.499 g (1.64 mmol) of saturated cardanol solubilized in 3.2 mL of CH₂Cl₂ (0.5 M), it was added 0.23 mL (1.97 mmol) of benzoyl chloride, 0.20 mL (2.46 mmol) of pyridine and an amount of catalytic DMAP. The solution was under agitation, at room temperature, for 30 minutes, when followed by CCD, the end of the reaction was verified. The mixture was diluted in CH₂Cl₂ (15 mL) and washed six times with water (6×20 mL), once with 5% of a HCL solution (15 mL), until the aqueous phase could reach a pH=1, once with saturated solution of NaHCO₃ (15 mL) and once with brine (20 mL). The organic phase was dried under Na₂SO₄ and the solvent evaporated to originate a product with 97% of yield.

Cardanolyla Benzoate—97% Yield

IR (KBr) υₘₐₓ: 2921, 2848, 1731, 1610, 1586, 1488, 1463, 1451, 1265, 1173, 1146, 1064 cm⁻¹.

¹H NMR (200 MHz, CDCl₃): δ: 0.8-1.0 (m, 3H); 1.2-2.0 (m, 26H); 2.5-2.7 (m, 2H); 7.0-7.8 (m, 7H); 8.1-8.5 (m, 2H).

### EXAMPLE 9

### Reactions of the Saturated Cardanol O-Alkylation

### General Procedure

In a flask containing 1.102 g (3.62 mmol) of saturated cardanol, solubilized in 18.0 mL of CH₂Cl₂ (0.2 M), it was added 5.0 mL of a NaOH 3 M solution, 10 drops of Aliquat® 336 and, under constant agitation, 1.2 mL (12.68 mmol) of (CH₃)₂SO₄. The two-phase solution stood at room temperature for 30 minutes when, followed by CCD, the end of the reaction was verified. The mixture was diluted in CH₂Cl₂ (30 mL) and washed once with water (30 mL), once with NH₄OH 2 M solution (25 mL) and twice with brine (2×30 mL). The organic phase was dried under Na₂SO₄ and the solvent evaporated to obtain, after a vacuum distillation to remove the dimethyl sulfate excess, the methylated product (oil).

3-pentadecyl-1-methoxycardanol—97% Yield

IR (film) υₘₐₓ: 2923, 2854, 1601, 1585, 1487, 1466, 1260, 1152, 1048 cm⁻¹.

¹H NMR (200 MHz, CDCl₃): δ: 0.8-1.0 (m, 3H); 1.2-1.8 (m, 26H); 2.5-2.7 (m, 2H); 3.8 (s, 3H); 6.6-6.8 (m, 3H); 7.1-7.3 (m, 1H).

### EXAMPLE 10

### Reactions of Friedel—Crafts Acylation

### General Procedure

In a flask containing 1.264 g (3.97 mmol) of the protected cardanol (obtained in the previous stage) solubilized in 8.0 mL (0.5 M) of distilled nitrobenzene, it was added 4.77 mmol of benzoyl chloride and 5.09 mmol of AlCl₃. The solution was put in bain-marie (T˜50-60° C.), and it was coupled, to the flask, a reflux condenser and to this a hose to collect HCl gaseous dived in a beaker with water. The solution was under heating and agitation for 2 hours and 30 minutes, when the HCl evolution was ceased. Then, it was spilled to a mixture of 1.7 mL concentrated HCl and pricked ice. To the reaction mixture 30 mL of ethyl ether was added and it was washed once with 5% of NaOH solution (30 mL), twice with water (2×30 mL) and once with brine (30 mL). The organic phase was dried under anhydrous Na₂SO₄, the evaporated solvent and the product was vacuum distilled to remove the nitrobenzene. The final product was columned to provide a mixture of isomers from the saturated cardanol, with a total yield of 70%.

4-acetylcardanol—70% Yield

IR (film) υₘₐₓ: 2924, 2853, 1660, 1603, 1567, 1494, 1464, 1377, 1269 cm⁻¹.

¹H NMR (200 MHz, CDCl₃): δ: 0.8-1.0 (m, 3H); 1.2-1.3 (m, 24H); 1.5-1.6 (m, 2H); 2.7 (dd, J=7.7 e 7.8 Hz, 2H); 3.9 (s, 3H); 6.7 (dd, J=8.5 e 2.6 Hz, 1H); 6.8 (d, J=2.6 Hz, 1H); 7.3 (d, J=8.5 Hz, 1H); 7.4-7.6 (m, 3H); 7.7-7.8 (m, 2H).

¹³C NMR (50 MHz, CDCl₃): δ: 14, 23, 30, 32, 34, 56, 110, 116, 128, 130, 131, 132, 133, 139, 146, 162, 198.

2-acetylcardanol—31% Yield

IR (film) υₘₐₓ: 2924, 2853, 1660, 1603, 1567, 1494, 1464, 1377, 1269 cm⁻¹.

¹H NMR (200 MHz, CDCl₃): δ: 0.8-1.0 (m, 3H); 1.2-1.3 (m, 24H); 1.5-1.6 (m, 2H); 2.7 (dd, J=7.7 e 7.8 Hz, 2H); 3.9 (s, 3H); 6.7 (dd, J=8.5 e 2.6 Hz, 1H); 6.8 (d, J=2.6 Hz, 1H); 7.3 (d, J=8.5 Hz, 1H); 7.4-7.6 (m, 3H); 7.7-7.8 (m, 2H).

### EXAMPLE 11

### Reactions of CNSL Unsaturated Phenolic Derivative Ozonolysis

### General Procedure

A solution containing diacetylcardols (9.6 mmol) in dichloromethane (50 mL) and methanol (50 mL) to −70° C. was treated with an ozone flow during 2 hours, which end was followed by thin layer chromatography. Then, the reaction mixture was espurged with nitrogen and to this a 4 g of sodium borohydride was added, staying the mixture under agitation for 14 hours. After the addition of water, the reaction mixture was hydrolyzed with 10% of hydrochloric acid and then extracted with ethyl acetate (3×40 mL). The organic phase were washed with brine, dried under sodium sulfate and, after the solvent removal under reduced pressure, it was obtained a white solid with 87% of yield, b.p. 108-110° C.

IR (KBr) υₘₐₓ: 3500-2800, 2956, 2851, 1598, 1520, 1512, 1158, 1071, 1048 cm⁻¹.

¹H NMR (300 MHz, CD₃COCD₃): δ: 1.31 (br, 8H, CH₂O); 1.51 (m, 4H, CH₂); 2.43 (t, 2H, ArCH₂); 3.56 (t, 2H, CH₂O); 3.81 (br, OH); 6.17 (m, 3H, ArH); 8.10 (s, 2H, OH);

¹³C NMR (75 MHz, CD₃COCD₃): δ: 25.8; 29.1; 29.3; 31.2; 32.8; 35.7; 62.6; 100.0; 107.7 (2C); 145.8; 159.2.

### EXAMPLE 12

### Obtention of the 2,4-dihydroxy-6-(8-hydroxyoctyl)benzaldehyde Derivative—Gattermann Reaction

### General Procedure

To a three-neck flask, adapted with a reflux condenser, a PVC tube for gas boiling and an exit for trap with sodium hydroxide, it was added 1.0 g of 2,4-dihydroxy-6-(8-hydroxyoctyl)benzene (4.2 mmol) solubilized in THF (5 mL), anhydrous diethylic ether (100 mL) and 1.24 g of anhydrous zinc cyanide (10.5 mmol). The mixture was kept under vigorous magnetic agitation during 20 to 30 minutes, while gaseous hydrochloric acid was bubbled in solution until the complete solubilization of the zinc cyanide. The reaction proceeded with the HCl bubbling until it was completed in 1.5 hours. The intermediary imidic was separated from the solvent through filtration and hydrolyzed with 20% of chloridric acid, under heating, for 2 hours. After cooling at room temperature, the reaction mixture was extracted with ethyl acetate (3×40 mL) and the organic phase washed with brine and dried in sodium sulfate. After the solvent evaporation in a reduced pressure, it was obtained a solid, which after purification in silica gel column eluded with hexane-ethyl acetate 3:1 provided the expected yield of 85%, b.p. 68-71° C.

IR (KBr) υₘₐₓ: 3125, 2932, 2853, 1615, 1500, 1312, 1264, 1202, 1162, 1058 cm⁻¹.

¹H NMR (300 MHz, CD₃COCD₃): δ: 1.1-1.8 (br, 12H, CH₂); 2.86 (t, 2H, ArCH₂O); 3.53 (t, 2H, CH₂O); 3.91 (br, OH); 6.16 (s, 1H, ArH); 6.30 (s, 1H, ArH); 10.0 (s, CHO); 12.51 (s, 2H, ArOH).

¹³C NMR (75 MHz, CD₃COCD₃): δ: 26.6; 28.9; 29.9; 31.7; 32.9; 33.0; 61.9; 101.0; 110.2 (2C); 112.3; 150.5; 165.7; 166.8; 193.5.

### EXAMPLE 13

### Cardanyl Benzoate Fries Rearrangement

### General Procedure

In an Erlenmeyer (50 mL) it was added 0.1 g (0.2459 mmol) of cardanyl benzoate, 0.333 g (2.5 mmol) of anhydrous aluminum chloride and 0.5 of chlorobenzene. The mixture was submitted to microwaves radiation, potency 10 (950 Watts), for 10 minutes. After cooling at room temperature, a HCl 6 M solution was added (2 mL) and the mixture was extracted with dichloromethane (3×15 mL). The organic phase were washed with brine, dried under sodium sulfate and concentrated to the reduced pressure. After purification in silica gel chromatographic column, eluded with hexane:dichloromethane 2:1 it was obtained a primrose-yellow solid with a 70% yield, characterized as 2-benzoylcardanol

¹H NMR (300 MHz, CD₃COCD₃): δ: 1.1-1.8 (br, 12H, CH₂); 2.86 (t, 2H, ArCH₂O); 3.53 (t, 2H, CH₂O); 3.91 (br, OH); 6.16 (s, 1H, ArH); 6.30 (s, 1H, ArH); 10.0 (s, CHO); 12.51 (s, 2H, ArOH).

¹³C NMR (75 MHz, CD₃COCD₃): δ: 26.6; 28.9; 29.9; 31.7; 32.9; 33.0; 61.9; 101.0; 110.2 (2C); 112.3; 150.5; 165.7; 166.8; 193.5.

Phototoxicity Test in Saccharomyces cerevisiae Yeast

The method applied was used by Freitas (Freitas, Z. M. F., I Ax, P. A., Dellamora-Ortiz, G. M., Santos, E. P., Gonçalves, J. C. S., S.T.P Pharma Sciences, 2000, 10 (3): 239-242) for the Saccharomyces cerevisiae yeast use, wild type strain D273-10B, at room temperature, which dense cell layer is not sensitive to the ultraviolet radiation among 320-390 nm (UVA), besides being innocuous.

The yeast growth was in a YPD medium, constituted by yeast extract (1%), peptone (2%), anhydrous glucose (2%), agar (2%).
In the test, the 8-methoxypsoralen 0.1 g % solution was used as phototoxic standard, and the octyl methoxycinnamate sunscreen (0.1 g %) was used as reference for the phototoxicity absence, ethanol was used as solvent. The studied substances were applied in 1 g % concentration in Wharman no. 1 sterile filter paper disks, and fixed on the surface of culture media plates.
A S. cerevisiae suspension was prepared in sterilized water (10 mL). Aliquots of 0.2 mL were applied and spread in the culture plates using a glass loop. Two plates were prepared for each sample. After seeding and applying the samples, one plate was allowed to grow under two UVA lamps (320-390 nm). A control plate was grown in the dark.
For the result analysis the following aspects were observed:
The presence of a clear zone around the test substance in the light and the its absence in the darkness indicate the sample phototoxicity;
The absence of a clear zone around the test substance in the light and in the darkness indicate that the sample is not phototoxic (Freitas, Z. M. F., I Ax, P. A., Dellamora-Ortiz, G. M., Santos, E. P., Gonçalves, J. C. S., 2000, S.T.P Pharma Sciences, 10 (3) 239-242).

Those results are summarized in the table I below.

TABLE I
Saccharomyces cerevisiae growth in the presence of different
substances under fluorescent light and in the darkness.

| Substance | Darkness* | UV light* |
|---|---|---|
| 8-methoxypsoralen | Absence | Presence |
| Parsol | Absence | Absence |
| V1 | Absence | Absence |
| V2 | Absence | Absence |
| V3 | Absence | Absence |
| V4 | Absence | Absence |
| V5 | Absence | Absence |
| V6 | Absence | Absence |
| V7 | Absence | Absence |
| V8 | Absence | Absence |
| V9 | Absence | Absence |
| V10 | Absence | Absence |
| V11 | Absence | Absence |
| V12 | Absence | Absence |
| V13 | Absence | Absence |
| V14 | Absence | Absence |
| V15 | Absence | Absence |
| V16 | Absence | Absence |
| V17 | Absence | Absence |
| V19 | Absence | Absence |
| V20 | Absence | Absence |
| V21 | Absence | Absence |
| V23 | Absence | Absence |
| V24 | Absence | Absence |
| V25 | Absence | Absence |
| V26 | Absence | Absence |
| V27 | Absence | Absence |
| V28 | Absence | Absence |
| V30 | Absence | Absence |
| V31 | Absence | Absence |
| V32 | Absence | Absence |
| V33 | Absence | Absence |
| V34 | Absence | Absence |
| V35 | Absence | Absence |
| V36 | Absence | Absence |
| V37 | Absence | Absence |

*Absence or presence of a clear zone around the disk containing the test substance after the growth under UV light and in the darkness.

### Absorption in the Ultraviolet

The samples were diluted in 10 ug/mL ethanol and it was verified, by spectrophotometric method, their absorptions in the ultraviolet wavelength, being determined the values of A^{1%}_{1cm}. A good A^{1%}_{1cm} value usually has 3 numbers after the comma, and such values are shown in the Table II below.

TABLE II
Compound absorption band

| Substance | λ | Abs peak | 1%1 cm | Solvent |
|---|---|---|---|---|
| V1 | 276 | 0.047 | 47 | Ethanol |
| V2 | 275 | 0.022 | 22 | Ethanol |
| V3 | 275 | 0.047 | 47 | Ethanol |
| V4 | 274 | 0.029 | 29 | Ethanol |
| V5 | 275 | 0.046 | 46 | Ethanol |
| V6 | 270 | 0.015 | 15 | Ethanol |
| V7 | 274 | 0.057 | 57 | Ethanol |
| V8 | 272 | 0.016 | 16 | Ethanol |
| V9 | 302 | 0.064 | 64 | Ethanol |
| V10 | 301 | 0.031 | 31 | Ethanol |
| V11 | 280 | 0.051 | 51 | Ethanol |
| V12 | 305 | 0.088 | 88 | Ethanol |
| V13 | 304 | 0.057 | 57 | Ethanol |
| V14 | 280 | 0.062 | 62 | Ethanol |
| V15 | 299 | 0.077 | 77 | Ethanol |
| V16 | 302 | 0.070 | 70 | Ethanol |
| V17 | 275 | 0.053 | 53 | Ethanol |
| V19 | 275 | 0.050 | 50 | Ethanol |
| V20 | 282 | 0.708 | 708 | Ethanol |
| V21 | 285 | 0.029 | 29 | Ethanol |
| V23 | 266 | 0.316 | 316 | Ethanol |
| V24 | 221 | 0.499 | 499 | Ethanol |
| V25 | 280 | 0.051 | 51 | Ethanol |
| V26 | 280 | 0.077 | 77 | Ethanol |
| V27 | 292.5 | 0.101 | 101 | Ethanol |
| V28 | 245 | 0.283 | 283 | Ethanol |
| V29 | 276 | 0.421 | 421 | Ethanol |
| V30 | 245 | 0.098 | 98 | Ethanol |
| V31 | 229 | 0.5865 | 586 | Ethanol |
| V32 | 294 | 1.088 | 1088 | Chloroform |
| V33 | 309.6 | 0.4816 | 418 | Ethanol |
| V34 | 275.6 | 0.588 | 588 | Hexane |
| V35 | 309 | 0.350 | 350 | THF |
| V36 | 331 | 1.167 | 1167 | DMSO |
| V37 | 274 | 0.6415 | 642 | THF |

### SPF Determination in Vitro

Sun Protection Factor (SPF)

SPF is the UV energy requested to produce a minimum erythema dose (MED) in the protected skin (after product application of mg/cm²), divided by the UV energy requested to produce a minimum erythema dose in the non-protected skin (Factor, 1997, Cosmetic On Line, 105: 37-46).

SPFi=MED protected skins/MED non-protected skins; SPF=ΣSPFi/n

This relationship is verified by the analysis method in vivo in which it is used 20 healthy individuals, during 3 days for the results conclusion.

Aiming at being faster and getting a way of controlling the quality of pharmaceutical preparations containing sunscreens, there was a search for an in vitro method that had the spectrophotometry as main principle. This method uses the mathematical equation developed by Mansur, as shown (Mansur, J. S., Breder, M. N. R., Mansur, M. C. A., Azulay, R. D., 1986, An. Bras. Dermatol., 61: (3) 121-24).

SPF spectrophotometric ═FC. Σ EE (λ).I (λ).Abs (λ)

SPF=sun protection factor

FC=correction factor=10, in relation to the in vivo test

EE=eritemogenic effect of the solar radiation in each λ

I=solar radiation intensity in each λ

Abs=absorbance in each λ

Table III shows the ponderation used by Sayre in the calculation of SPF.

TABLE III
Ponderation used in the SPF calculation by spectrophotometry
(Sayre, R. M., Agin, P. P., Scans Vee, G. J., Marlowe, E.,
Photochem Photobiol, 1979, 29: 559-66)

| λ_(nm) | EE (λλ) normalized |
|---|---|
| 290 | 0.0150 |
| 295 | 0.0817 |
| 300 | 0.2874 |
| 305 | 0.3278 |
| 310 | 0.1864 |
| 315 | 0.0839 |
| 320 | 0.0180 |
| | 1.000 |

Table IV presents the results of the studies between chemical structures-photoprotector activity relations, making clear the wide spectrum of SPF values for the maximum concentration of 5 g % for each substance.

TABLE IV
SPF results for spectrophotometry for the V1-V37 derivatives in
a 5 g % maximum concentration

| Substance | Concentration | SPF |
|---|---|---|
| V1 | 4 g % | 0.064 |
| V2 | 5 g % | 0.450 |
| V3 | 1 g % | 0.025 |
| V4 | 1 g % | 0.029 |
| V5 | 5 g % | 0.047 |
| V6 | 5 g % | 0.061 |
| V7 | 5 g % | 0.027 |
| V8 | 5 g % | 0.000 |
| V9 | 5 g % | 0.610 |
| V10 | 5 g % | 0.390 |
| V11 | 4 g % | 0.093 |
| V12 | 5 g % | 0.930 |
| V13 | 5 g % | 0.660 |
| V14 | 5 g % | 0.720 |
| V15 | 5 g % | 0.740 |
| V16 | 1 g % | 0.150 |
| V17 | 1 g % | 0.000 |
| V19 | 5 g % | 0.000 |
| V20 | 5 g % | 0.900 |
| V21 | 5 g % | 0.000 |
| V23 | 5 g % | 0.530 |
| V24 | 5 g % | 0.520 |
| V25 | 5 g % | 0.100 |
| V26 | 5 g % | 0.059 |
| V27 | 5 g % | 0.760 |
| V28 | 5 g % | 0.740 |
| V29 | 2.5 g % | 1.100 |
| V30 | 5 g % | 0.500 |
| V31 | 5 g % | 0.000 |
| V32 | 5 g % | 9.500 |
| V33 | 5 g % | 5.200 |
| V34 | 5 g % | 1.700 |
| V35 | 5 g % | 3.300 |
| V36 | 5 g % | 7.700 |
| V37 | 5 g % | 1.100 |

### EXAMPLE 14

### Mutagenicity

The solar light, especially UV, can cause damages to DNA and, therefore, lead to carcinogenic and mutagenic events. The skin protection for the solar radiation reflection (physical filters) or absorption for sunscreens (chemical filters) are preventive measures against such toxic effects (Utesch, D., Splittgerber, J., 1996, Mutation Res., 361, 41-8). The possibility, however, is in the fact that the solar light can excite the absorbent molecules (i.e. sunscreens), and transform them in intermediary reactives (i.e. free radicals) that can damage DNA, which, in turn, is dangerous (Utesch, D., Splittgerber, J., 1996, Mutation Res., 361, 41-8; Knowland, J., Mckenzie, E. A., Mchugh, P. J., et al., 1993, FEBS, 324: (3) 309-13).

Concerning the issue, the scientific committee on cosmetology of The European Commission published guidelines in 1982 saying that the phototoxicity, photomutagenecity, photosensitivity studies are requested for certain cosmetic ingredients, in which the chemical structure indicates a possible danger. In some cases, as with the sunscreens, such studies should be made, because the risk is higher due to the way they are used (Knowland, J., Mckenzie, E. A., Mchugh, P. J., et al., 1993, FEBS, 324: (3) 309-13).
1. For mutagenecity tests is understood all those that detect alterations in the genetic material. If these alterations aren't repaired or they are inadequated repaired, it is said that there is a mutation (Splenger, J.; Bracher, M.; Weide, J., 1990, Cosmetics & toiletries, 2, 18-23). The most frequently used test, to verify gene mutations, is the Ames method (Maron, D. M., Ames, B., 1983, Mutation Research, 113, 173-215).

Methodology

AMES TEST (Maron, D. M., Ames, B., 1983, Mutation Research, 113, 173-215)

Vogel-Bonner E Médium (VBEM)

| | | |
|---|---|---|
| Magnesium sulfate (MgSO₄•7H₂O) | | 10 g |
| Citric acid (H₃C₆H₅O₇•H₂O) | | 100 g |
| Potassium phosphate (K₂HPO₄•3H₂O) | | 500 g |
| Sodium ammonium phosphate (Na(NH₄)HPO₄•4H₂O) | | 175 g |
| Distilled water (45 degrees) | qsp | 1000 mL |

p.s.:
to distribute in several flasks and autoclave it to 120 degrees for 20 min

VBEM in Plates

| | |
|---|---|
| Agar Difco | 9 g |
| H₂O | 558 mL |
| VBEM (50X) | 12 mL |
| Glucose 40% | 30 mL |
| to autoclave | |

Surface Gelose

| | |
|---|---|
| Agar Difco | 0.6% |
| NaCl | 0.5% |

Obs: after sterilization add 10 mL of L-histidine/D-biotin mixed solution to each 100 mL of surface gel

Mixed Solution

| | |
|---|---|
| L-histidine monohydrate monochlorhydrate | 11 mg |
| D-biotin | 12.36 mg |
| Sterile distilled water | 100 mL |

p.s.:
filter sterilization in a 0.45 micron milipore filter

Procedure

The used strains were: TA 98, TA 99, TA101, TA102.

The 4-nitroquinoline 1-oxide (4NQO) solution was used as genotoxicity pattern.

The samples were diluted in 5% of tetrahydrofuran (THF). Two aliquots were removed and put in glass flasks, and irradiated with 20 kJ/m² (27 J/m²/s for 12′34″) of UVA radiation and 10 kJ/m2 (7.8 J/m²/s for 21′36″) of UVB, to verify the photomutagenicity.

Results

The samples that presented the best SPF values, V32, V33, V34, V35, V36, V37 were selected because these molecules presented the ideal characteristics to be considered new sunscreens.

These samples were tested through the Ames method, in the 5% concentration in THF, using the TA98, TA99, TA101, TA102 strands. It was applied directly to the plates 10 μL of each sample, without irradiation, and after UVA (20 kJ/m²) and UVB (10 kJ/m²) irradiation, and they didn't demonstrate to be mutagenic or photomutagenics (n=3). The non-irradiated samples didn't demonstrate mutagenicity when compared to the positive pattern for this test, 4NQO. When the samples were irradiated with UVAr and UVBr, they didn't show a photomutagenic answer either (n=3).

The used solvent, THF, was tested alone and it didn't demonstrate mutagenic answer.

### EXAMPLE 15

### Genotoxicity

Genotoxicity tests can be defined as tests in vitro and in vivo, designated to detect compounds that induce direct or indirect genetic damages by several mechanisms. These tests should be able to identify a danger with regard to the DNA damage and its fixation. The damage fixation at the DNA level in the form of genetic mutation, chromosome harm to a large extent, chromosome numeric and recombinant changes are usually considered essential for hereditary effects in the malignancy process. Compounds that generate genotoxic answers in tests that detect such damages types have potential to be considered carcinogenic and/or mutagenic for humans and, thus, induce cancer or hereditary effects (Ptitsyn, L. R.; Horneck, G.; Komova, O.; Kozubek, S.; Krasavin, E. A.; Bonev, M.; Rettberg, P., 1997, Applied and environmental microbiology, 63: (11) 4377-84).

a) SOS Spot Test

For the SOS spot test the production and induction of the β-galactosidase by the tester strain may be evidenced indicator plates containing a substrate: Xgal (5-bromine-4-chlorine-3-indolyl-β-D-galactoside), which releases a blue coloration when hydrolysed for the β-galactosidase. The simplicity of the SOS Chromotest on plate (SOS spot test) permits several samples to be tested at the same time (Quillardet, P.; Hofnung, M., 1985, Mutation Research, 147, 65-78).

Material
Strand cultures of E. coli PQ 35 and PQ 37
Solid and liquid culture media
Buffers and solutions
UVA and UVB lamps

Procedure

Cultivation the PQ35 and PQ37 strands in LB-Amp (20 ug/ml) overnight.

Replication 0.25 mL of each strand in 10 mL of LB-Amp and cultivate until the exponential phase (108 cels/mL)

Flow, 100 μL of each culture, with the help of 3 mL of TopAgar, on the plates containing half M63 added with Xgal and to let it dry (10 min)

Drop 10 μL of the agent to be tested on the culture in the plate and let it dry (20 to 40 min)

Put in the greenhouse at a temperature of 37° C. overnight

The following day to verify the appearance or not of the blue halo.

The samples were diluted in 5% of THF. Two aliquots were removed, put in glass flasks, and irradiated with 20 kJ/m² (27 J/m²/s for 12′34″) of UVA radiation and 10 kJ/m² (7.8 J/m²/s for 21′36″) of UVB, to verify the photomutagenicity.

### Results

For the SOS spot test, the samples were applied directly in the plate containing the culture medium; before applying it in the plates, two brackets of the samples were irradiated with UVA and UVB radiation, respectively, to evaluate the photogenotoxic potential of the substances.

The nitroquinoline 1-oxide (4NQO) solution was used as genotoxicity pattern. The THF solvent was tested alone and it didn't demonstrate to be genotoxic.

The irradiated and non-irradiated samples with UVA and UVB radiation, V33, V35, and V37 (to 5% in THF) didn't present blue halo for PQ35 and PQ37, indicating they weren't genotoxics in the tested concentration (n=3).

The V32, V34 and V36 samples (to 5% in THF) presented a light blue halo just for PQ37, when irradiated with UVA and UVB radiation, they demonstrated a light genotoxicity and cytotoxicity for both strands (n=3). Therefore it was made a quantification of this supposed genotoxicity through the SOS chromotest.

b) SOS Chromotest in E. coli PQ37

The SOS chromotest was described by Quillardet & Hofnung (Pasteur Institute Pasteur, Paris) in 1982 (Quillardet, P.; Huisman, O.; D'ari, R.; Hofnung, M. 1982, Proc. Acad. Sci., 79, 5971-5975) as an alternative for the Ames test and it is based on the application of selected strands of Escherichia coli PQ37 to detect damages in DNA. This is one of the fastest and simplest tests for genotoxines. (Bombardier, M., Bermingham, N., Legault, R., Fouquet, A., 2001, Chemosphere, 42, 931-944; Kevekordes, S., Mersch-Sundermann, V., Burghaus, C. M., Spielberger, J., Schmeiser, H. H., Arlt, V. M., Dunkelberg, H., 1999, Mutation research, 445, 81-91).

Procedure
Cultivate E. coli PQ35 or PQ37 in LB-Amp (20 μg/mL) 10 ml overnight.
Repicate 0.2 mL of the culture in 10 mL of LB and cultivate until the exponential phase (approx. 108 cels/mL) for 2 hours and 30 minutes.
Dilute 1 mL of the culture in 9 mL of LB
Distribute 0.6 mL of the culture on the test tubes containing 20 μL of each substance to be tested.
Incubate on the shaker at 37° C. for 2 hours.
To divide the cultures in series: X AND Y
Series X (β-gal)
Take out 300 μL and join it to 2.7 mL of B buffer.
Incubate at 37° C. in the bath for 10 minutes
Add 0.6 mL of ONPG (4 mg/mL of pH 7.0 TF) and write the time down
When it colors (10 to 90 min) add 2 ml of Na₂CO₃ 1 M

Series Y—Alkaline Phosphatase
Take out 300 μL and add it to 2.7 mL of P buffer
Incubate at 37° C. in the bath for 10 minutes
Add 0.6 mL of PNPP (4 mg/ml of pH 7.0 TF) and write the time down
When it colors (10 to 90 minutes) add 1 ml of HCl 2.5 M
5 minutes later, add 1 mL of the Tris 2 M solution

Calculation:

The activities of the β-galactosidase (β-gal) and alkaline phosphatase (AF) are calculated as the absorbance value to 405 nm times 1000, divided by the test time (Kevekordes, S., Mersch-Sundermann, V., Burghaus, C. M., Spielberger, J., Schmeiser, H. H., Arlt, V. M., Dunkelberg, H., 1999, Mutation research, 445, 81-91).

First the rate between Rx and Ro, that are the β-gal or alkaline phosphatase activities of the substance in the concentration “x” (Rx) and in the concentration zero (Ro). To calculate the induction factor (IF):

FI=Rx/Ro(β-gal)/Rx/Ro(AF)

### Results

Aiming at a quantification of the supposed genotoxicity presented in SOS Spot test for V32, V34 and V36, in the concentration of 5% in THF, it was performed the SOS Chromotest (table 4).

TABLE V
Genotoxic activity of the V32, V34, V36, octyl p-
methoxycinnamate (PMCO) substances in culture of E. coli
(PQ37) (n = 3)

| COMPOUND | DOSE g % | Unit AF | Unidades β-gal | IF |
|---|---|---|---|---|
| V32 | 0 | 0.067 | 0.551 | 0.424 |
| | 1 | 0.121 | 0.376 | 0.375 |
| | 2.5 | 0.151 | 0.612 | 0.492 |
| | 4 | 0.076 | 0.464 | 0.739 |
| | 5 | 0.092 | 0.372 | 0.489 |
| | 10 | 0.096 | 0.235 | 0.296 |
| V34 | 0 | 0.067 | 0.551 | 0.424 |
| | 1 | 0.124 | 0.784 | 0.765 |
| | 2.5 | 0.077 | 0.725 | 1.136 |
| | 4 | 0.229 | 0.772 | 0.407 |
| | 5 | 0.201 | 0.797 | 0.479 |
| | 10 | 0.155 | 0.861 | 0.674 |
| V36 | 0 | 0.075 | 1.599 | 1.097 |
| | 1 | 0.079 | 0.972 | 0.577 |
| | 2.5 | 0.053 | 0.773 | 0.677 |
| | 5 | 0.059 | 0.154 | 0.123 |
| PMCO | 0 | 0.067 | 0.551 | 0.424 |
| | 1 | 0.078 | 0.753 | 1.16 |
| | 2.5 | 0.241 | 0.647 | 0.32 |
| | 4 | 0.0493 | 0.488 | 1.19 |
| | 5 | 0.086 | 0.614 | 0.85 |
| | 10 | 0.080 | 0.639 | 0.966 |

FI = induction factor

A compound is classified as “not genotoxic” if the induction factor remains <1.5, as “marginal” if the induction factor is between 1.5 and 2.0, and as “genotoxic” if the induction factor exceeds 2.0 (Kevekordes, S., Mersch-Sundermann, V., Burghaus, C. M., Spielberger, J., Schmeiser, H. H., Arlt, V. M., Dunkelberg, H., 1999, Mutation research, 445, 81-91).

The V32, V34 and V36 substances, in a concentration varying from 1% to 10%, presented induction factors smaller than 1.5, being their results compared to the octyl p-methoxycinnamate, a very used sunscreen, demonstrating that it they are not genotoxic.

### EXAMPLE 16

### Phototoxicity

Phototoxicity is the term used to characterize the sharp reaction that it can be induced by an only application of the chemical product to the skin, associated to the ultraviolet or visible radiation exposition (ANVISA, 2003 guia para avaliação de segurança de produtos cosmético. http://www.anvisa.gov.br accessed in Feb. 20, 2004; Freitas, Z. M. F., I Ax, P. A., Dellamora-Ortiz, G. M., Santos, E. P., Gonçalves, J. C. S., 2000, S.T.P Pharma Sciences, 10 (3) 239-242; Dinardo, J. C., Wolf, B. A., Morris, W. E., Tenenbaum, S., Schnetzinger, R. W., 1985, J. Soc. Cosmet. Chem., 36, 425-433).

The tests use animals to evaluate “primary cutaneous phototoxicity”: guinea pig, rabbits, rats or mice. In spite of the protocols pattern publication concerning to phototoxicity tests in animals, no test was accepted by OECD (Organisation for Economic Co-operation and Development—nongovernmental organization headquartered in Paris). On the contrary, OECD recommends phototoxicity tests in vitro before testing in animals (Spielmann, H., Balls, M., Dupuis, J., Pape, W. J., Pechovitch, G., De Silva, O., Holzhütter, H.-G., Clothier, R., Desolle, P., Gerberick, F., Liebsch, M., Lovell, W. W., Maurer, T., Pfannenbecker, U., Potthast, J. M., Csato, M., Sladowski, D., Steiling, N., Brantom, P., 1998, Toxicology in vitro, 12, 305-327).

a) Phototoxicity in Vitro

In this work, the applied method was used by Freitas (Freitas, Z. M. F., I Ax, P. A., Dellamora-Ortiz, G. M., Santos, E. P., Gonçalves, J. C. S., 2000, S.T.P Pharma Sciences, 10 (3) 239-242) and also described by DiNardo (Dinardo, J. C., Wolf, B. A., Morris, W. E., Tenenbaum, S., Schnetzinger, R. W., 1985, J. Soc. Cosmet. Chem., 36, 425-433) for the use of the Saccharomyces cerevisiae yeast, wild strand D273-10B. This microorganism presents a good growth at room temperature, it forms a very thick cell layer, it is not sensitive to the ultraviolet radiation between 320-390 nm (UVA), besides being innocuous.

The yeast growth was in a YPD medium (TENAN, M.N., 1985), constituted by yeast extract (1%), peptone (2%), anhydro glucose (2%), agar (2%).

In the test, the 8-methoxypsoralen 0.1 g % solution was used as phototoxic pattern (Spielmann, H., Balls, M., Dupuis, J., Pape, W. J., Pechovitch, G., De Silva, O., Holzhütter, H.-G., Clothier, R., Desolle, P., Gerberick, F., Liebsch, M., Lovell, W. W., Maurer, T., Pfannenbecker, U., Potthast, J. M., Csato, M., Sladowski, D., Steiling, N., Brantom, P., 1998, Toxicology in vitro, 12, 305-327), and the octyl methoxycinnamate sunscreen (0.1 g %) was used as reference for the phototoxicity absence, ethanol was used as solvent. The substances in study were applied in the plate together with the reference pattern.

A S. cerevisiae suspension was prepared in sterilized water (10 mL). Aliquots of 0.2 mL were applied and spread in the culture plates using a glass loop. Two plates were prepared for each sample. After seeding and applying the samples, one plate was allowed to grow under two UVA lamps (320-390 nm). A control plate was grown in the dark.

For the result analysis the following aspects were observed:
The presence of a clear zone around the test substance in the light and the its absence in the darkness indicate the sample phototoxicity;
The absence of a clear zone around the test substance in the light and in the darkness indicate that the sample is not phototoxic (Freitas, Z. M. F., I Ax, P. A., Dellamora-Ortiz, G. M., Santos, E. P., Gonçalves, J. C. S., 2000, S.T.P Pharma Sciences, 10 (3) 239-242).

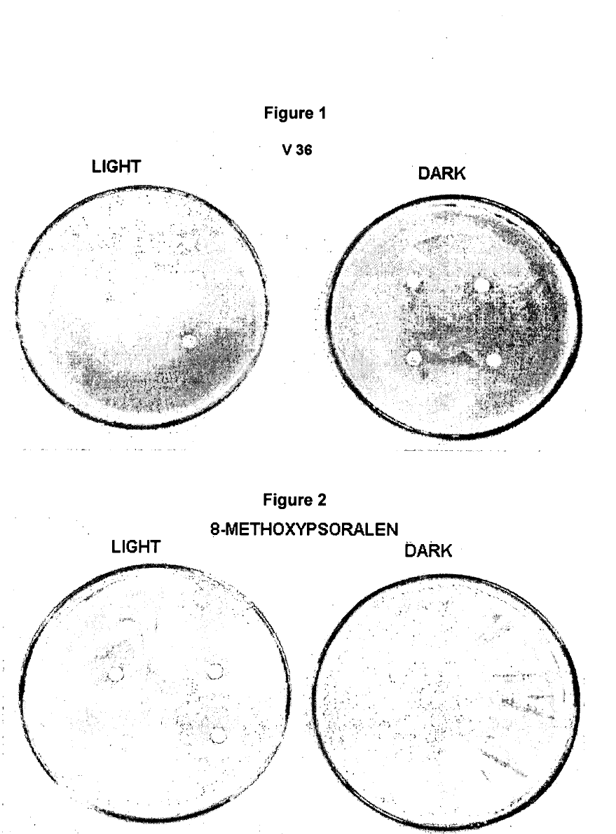

None of the 36 tested substances (1 g %) presented growth inhibition halo in both plates (irradiated and with light absence) demonstrating they are not phototoxics (FIG. 1). The phototoxicity pattern, 8-methoxypsoralen presented growth inhibition halo in the light (FIG. 2); and the octyl p-methoxycinnamate didn't present growth inhibition halo.

b) Phototoxicity in Vivo

The tests are accomplished in short hair line albino Guinea pigs (n=4). Twenty-four hours before the application of the test substance to 5% [tween/ethanol/water (1:1:10)], the animals dorsal portion hair are chemically removed. Four application sites are chosen, at random: 2 areas for the test substance and other 2 for the methoxypsoralen phototoxic standard at 0.1 g %, following by the UVA radiation exposition, and a control area is protected from the light. After 24 and 48 hours, the observations are made as for the erythema edema formation (AGÊNCIA NACIONAL DE VIGILÂNCIA SANITÁRIA-Brazil, Guia para avaliaçãa de segurança de produtos cosméticos. 2003.; Freitas, Z. M. F., I Ax, P. A., Dellamora-Ortiz, G. M., Santos, E. P., Gonçalves, J. C. S., 2000, S.T.P Pharma Sciences, 10 (3) 239-242; Dinardo, J. C., Wolf, B. A., Morris, W. E., Tenenbaum, S., Schnetzinger, R. W., 1985, J. Soc. Cosmet. Chem., 36, 425-433).

The V33, V34 and V35 substances, at 5 g % concentration, presented degree 1 erythema and in the irradiated and non-irradiated areas (n=4) in guinea pigs. The V32 and V36 substances didn't present erythema or edema in the irradiated and non-irradiated areas (n=4). The octyl p-methoxycinnamate pattern was tested (n=2) and it didn't present erythema or edema in the irradiated and non-irradiated areas. The phototoxic pattern, 8-methoxypsoralen, presented degree 1 erythema in 24 hours, and degree 2 in 48 hours only in the irradiated area as shown in FIG. 3.

### EXAMPLE 17

### Ocular Irritation Test

Considering an estimation and evaluation of the toxic properties of a substance for use in cosmetics, perfumery, for ex., the determination of the irritating properties and/or corrosive effects on the eyes of mammals, constitutes an important initial stage to indicate the probable risks for eyes and conjunctiva exposition to a test substance (Brito, A. S. 1994, Manual de ensaios toxicológicos in vivo. Ed. UNICAMP, Campinas, S P; Vigliogila, P. A., Rubin, J., 1983, Reacciones adversas por cosmeticos. In: Cosmiatria: fundamentos científicos y técnicos. 2. ed. Buenos Aires; NATIONAL AGENCY OF SANITARY MONITORING (AGÊNCIA NACIONAL DE VIGILÂNCIA SANITÁRIA) (Brazil), 2003, Guia para avaliação de segurança de produtos cosméticos; Draize, J. H.; Woodward, G.; Calvery, H. O., 1944, J. Pharm. Exper. Therap., 82 (4) 377-390).

Ocular irritation is the production, on the eyes, of reversible alterations as a consequence of a test substance application on the ocular cavity (Brito, A. S. 1994, Manual de ensaios toxicológicos in vivo. Ed. UNICAMP, Campinas, S P; Vigliogila, P. A., Rubin, J., 1983, Reacciones adversas por cosmeticos. In: Cosmiatria: fundamentos científicos y técnicos. 2. ed. Buenos Aires; NATIONAL AGENCY OF SANITARY MONITORING (AGÊNCIA NACIONAL DE VIGILÂNCIA SANITÁRIA) (Brazil), 2003, Guia para avaliação de segurança de produtos cosméticos; Draize, J. H.; Woodward, G.; Calvery, H. O., 1944, J. Pharm. Exper. Therap., 82 (4) 377-390).

### Methodology

The test substance is applied, in an only dose, 0.1 mL at 5% [tween/ethanol/water (1:1:10)], on one of the eyes of each one of the experience animals (n=3): the not-treated eye of each animal serves as control for the test. The degree of the corrosive/irritant effects is evaluated in precise and established intervals to provide a complete evaluation of the product effects (Brito, A. S. 1994, Manual de ensaios toxicológicos in vivo. Ed. UNICAMP, Campinas, S P; Vigliogila, P. A., Rubin, J., 1983, Reacciones adversas por cosmeticos. In: Cosmiatria: fundamentos científicos y técnicos. 2. ed. Buenos Aires; NATIONAL AGENCY OF SANITARY MONITORING (AGÊNCIA NACIONAL DE VIGILÂNCIA SANITÁRIA) (Brazil), 2003, Guia para avaliação de segurança de produtos cosméticos; Draize, J. H.; Woodward, G.; Calvery, H. O., 1944, J. Pharm. Exper. Therap., 82 (4) 377-390).

Administration: the test substance should be instilled or applied inside one of the eyes conjunctival bag of each one of the experience animals, after the cautious lifting of the eyeball inferior eyelid. Soon afterwards both eyelids should be put together, still cautiously, for ten seconds to avoid the substance loss. The other eye, that didn't receive any treatment type, will serve as control.

The eyes are examined 24, 48 hours after the instilation. If no irritation is shown, the experience is finished; if the irritation appears, the next reading should be made seven days after the product application. An extended observation can be necessary to check the evolution of the ocular lesions in relation to their reversible or irreversible character. Another pertinent observation to the conjunctiva, iris and cornea is that in which, is mentioned, in the report, all of the noticed lesions. The degree of the ocular reaction must be registered for each animal in each exam (Brito, A. S. 1994, Manual de ensaios toxicológicos in vivo. Ed. UNICAMP, Campinas, S P; NATIONAL AGENCY OF SANITARY MONITORING (AGÊNCIA NACIONAL DE VIGILÂNCIA SANITÁRIA) (Brazil), 2003, Guia para avaliação de segurança de produtos cosméticos; Draize, J. H.; Woodward, G.; Calvery, H. O., 1944, J. Pharm. Exper. Therap., 82 (4) 377-390).

The quotation of the ocular irritations is subject to several interpretations. To promote homogeneity of the values and co-operate with the laboratories that interpret the ocular irritation observations, it is convenient to use the guide that quantifies each one of the ocular lesions of each one of the studied parts of the eyes, in other words, iris, cornea and conjunctiva, as described below:

### Ocular Lesion Quotation

TABLE VI
Cornea (opacity - density degree)

| Lesion | Value |
|---|---|
| Without ulceration nor opacity | 0 |
| Dispersed or diffuse opacity areas, eminently visible details of | 1 |
| the iris | |
| Easily discernible translucent area, eminently visible details of | 2 |
| the iris | |
| Nacarade areas, completely invisible details of the iris, | 3 |
| dimension of the pupil only discernible | |
| Opaque cornea, iris no discernible through the opacity | 4 |

TABLE VII
Iris (generalized inflammatory response)

| Lesion | Value |
|---|---|
| Normal | 0 |
| Deeper folds, congestion, tumefaction, moderate peri-corneal | 1 |
| hyperemia or injected conjunctivas. It doesn't matter which is | |
| happening of those symptoms, or a combination of them: | |
| the iris continues to answer to the light | |
| Reaction absence to the light, hemorrhage, outstanding | 2 |
| destruction of the tissue (each one of those symptoms | |
| or their group) | |

TABLE VIII
Conjunctiva (redness of the eyelid conjunctiva, bulb, cornea
and iris)

| Lesion | Valor |
|---|---|
| Normal coloration | 0 |
| Hyperemia of certain blood vessels (injected eyes) | 1 |
| Diffuse purple coloration, individual blood vessels hardly | 2 |
| discernible | |
| Red coloration widely distributed | 3 |
| Chemosis (eyelids and/or nictitant membrane without tumefaction | 4 |
| or sunk) | |

### Results

It wasn't observed any alteration in the cornea, iris and conjunctiva of the rabbits that were treated with the following substances (V32, V33, V34, V35, V36 to 5%) in study (n=3).

### EXAMPLE 18

### Dermal Irritation Test

Considering an estimation and evaluation of the toxic properties of a substance for use in cosmetics, preservatives or defensive to be tested, the determination for the irritating properties and/or for the corrosive effects on the skin of mammals constitutes an important initial stage to indicate the probable results on the human skin related to this substance (Brito, A. S. 1994, Manual de ensaios toxicológicos in vivo. Ed. UNICAMP, Campinas, S P).

Dermal irritation is the production, on the skin, of reversible inflammatory alterations due to the application of a test substance (Brito, A. S. 1994, Manual de ensaios toxicológicos in vivo. Ed. UNICAMP, Campinas, S P).

a) Primary Dermal Irritation

The objective of this test is to evaluate the irritation that a cosmetic can provoke after a single application in the normal or harmed skin (Vigliogila, P. A., Rubin, J., 1983, Reacciones adversas por cosmeticos. In: Cosmiatria: fundamentos científicos y técnicos. 2. ed. Buenos Aires).

### Methodology

Twenty-four hours before the application of the test substance, the albino rabbits dorsal part hair are dehaired (n=3). It is chosen, randomly, four application sites, two of the which should be submitted to the abrasion. This last procedure, however, should not produce damages or bleeding to the skin. (Brito, A. S. 1994, Manual de ensaios toxicológicos in vivo. Ed. UNICAMP, Campinas, S P; NATIONAL AGENCY OF SANITARY MONITORING (AGÊNCIA NACIONAL DE VIGILÂNCIA SANITÁRIA) (Brazil), 2003, Guia para avaliação de segurança de produtos cosméticos; Draize, J. H.; Woodward, G.; Calvery, H. O., 1944, J. Pharm. Exper. Therap., 82 (4) 377-390).

The substances should be applied [0.5 mL of the sample at 5%, using as the mixture polissorbate80/ethanol/water (1:1:10) as solvent on the gauze and put later on the skin. The area should be covered again with a gauze compress fixed by a hipoallergenic tape and an adhesive paper tape (Brito, A. S. 1994, Manual de ensaios toxicológicos in vivo. Ed. UNICAMP, Campinas, S P; NATIONAL AGENCY OF SANITARY MONITORING (AGÊNCIA NACIONAL DE VIGILÂNCIA SANITÁRIA) (Brazil), 2003, Guia para avaliação de segurança de produtos cosméticos; Draize, J. H.; Woodward, G.; Calvery, H. O., 1944, J. Pharm. Exper. Therap., 82 (4) 377-390).

The exposition time is four hours. At the end of this period, the test substance is removed and the area washed with water to eliminate their residues, in a way to not alter a existent answer or the epidermis integrity (Brito, A. S. 1994, Manual de ensaios toxicológicos in vivo. Ed. UNICAMP, Campinas, S P; NATIONAL AGENCY OF SANITARY MONITORING (AGÊNCIA NACIONAL DE VIGILÂNCIA SANITÁRIA) (Brazil), 2003, Guia para avaliação de segurança de produtos cosméticos; Draize, J. H.; Woodward, G.; Calvery, H. O., 1944, J. Pharm. Exper. Therap., 82 (4) 377-390).

The observation of the edema and erythema on the skin's animals are evaluated 24, 48 and 72 hours after removing the compresses. The cutaneous irritations are observed and registered all the time, following the Draize scale (Brito, A. S. 1994, Manual de ensaios toxicológicos in vivo. Ed. UNICAMP, Campinas, S P; NATIONAL AGENCY OF SANITARY MONITORING (AGÊNCIA NACIONAL DE VIGILÂNCIA SANITÁRIA) (Brazil), 2003, Guia para avaliação de segurança de produtos cosméticos; Draize, J. H.; Woodward, G.; Calvery, H. O., 1944, J. Pharm. Exper. Therap., 82 (4) 377-390).

b) Cumulative Dermal Irritation

In the case of the cumulative irritation test, the applications are made through a period of seven consecutive days, and the evaluations are made 24 and 48 hours after the last application (n=3) (NATIONAL AGENCY OF SANITARY MONITORING (AGÊNCIA NACIONAL DE VIGILÂNCIA SANITÁRIA) (Brazil), 2003, Guia para avaliação de segurança de produtos cosméticos).

Results

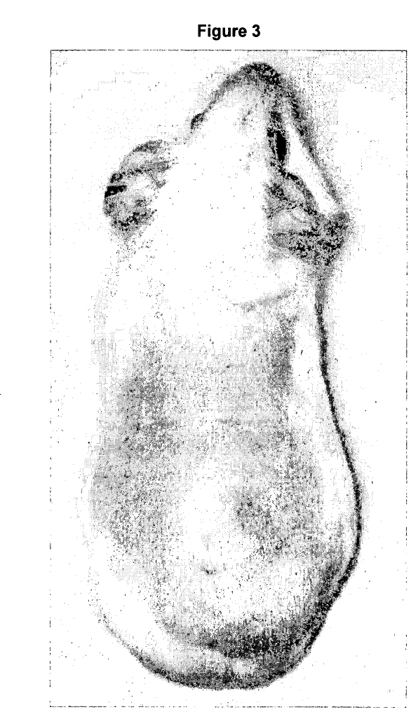

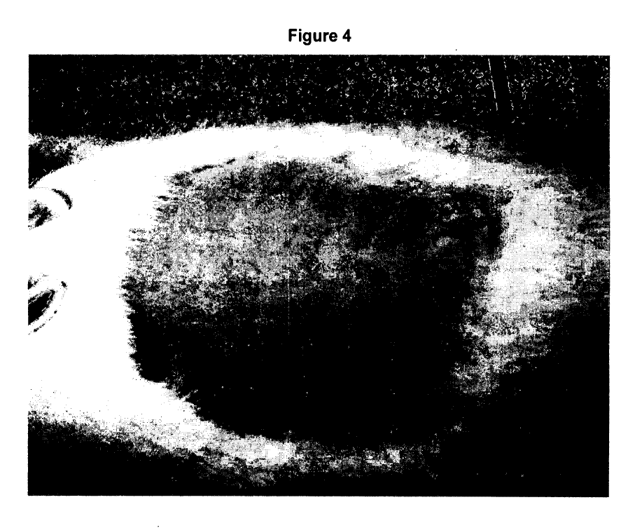

No substance in the concentration of 5% presented edema or erythema during the observed time in the normal and scraped areas as shown in the FIG. 4.

### EXAMPLE 19

### Molar Absorptivity

The absorptive characteristics of each molecule were evaluated through the determination of its molar absorptivity. The efficiency of the sunscreens in a certain wavelength is function of its molar absorptivity coefficient (ε). So, sunscreens that possess high ε values are more efficient in absorbing the energy of the ultraviolet radiation.

In the tables IX and X we have the molar absorptivity value of each substance in the wavelength where they obtained maximum absorption.

In the tables XI and XII, we have the molar absorptivity value of the substances at 305 nm, which represents the UVB intermediate wavelength (290-320 nm). And we can notice that the substances that presented the best values are 32, 33, 34, 35 and 36, indicating that they are very effective in absorbing the UVB radiation.

TABLE IX
Results of A1% 1 cm in the λ maximum and of molar absorptivity
in the respective wavelengths (λmax) from the substances
V1 to V19.

| Substance | λmax | A1%1 cm | PM (g/mol) | ε |
|---|---|---|---|---|
| V1 | 276 | 47 | 318.19 | 1495.5 |
| V2 | 275 | 22 | 407.21 | 895.9 |
| V3 | 275 | 47 | 320.19 | 1504.9 |
| V4 | 274 | 29 | 409.21 | 1186.7 |
| V5 | 275 | 46 | 302.20 | 1390.1 |
| V6 | 270 | 15 | 344.21 | 516.3 |
| V7 | 274 | 57 | 304.20 | 1733.9 |
| V8 | 272 | 16 | 346.21 | 553.9 |
| V9 | 302 | 64 | 346.19 | 2215.6 |
| V10 | 301 | 31 | 388.20 | 1203.4 |
| V11 | 280 | 51 | 374.21 | 1908.5 |
| V12 | 305 | 88 | 348.19 | 3064.1 |
| V13 | 304 | 57 | 390.20 | 2224.1 |
| V14 | 280 | 62 | 376.21 | 2332.5 |
| V15 | 299 | 77 | 334.05 | 2572.2 |
| V16 | 302 | 70 | 338.52 | 2369.6 |
| V17 | 275 | 53 | 304.43 | 1613.5 |
| V19 | 275 | 50 | 318.54 | 1592.7 |

TABLE X
Results of A1% 1 cm in the λmaximum and of molar absorptivity in
the respective wavelengths (λmax) from the substances V20 to V37

| Substance | λ max | A1%1 cm | PM (g/mol) | ε |
|---|---|---|---|---|
| V20 | 282 | 708 | 423.66 | 29995.1 |
| V21 | 285 | 29 | 361.59 | 1048.6 |
| V23 | 266 | 316 | 408.61 | 12912.1 |
| V24 | 221 | 499 | 374.56 | 18690.5 |
| V25 | 280 | 51 | 376.58 | 1920.6 |
| V26 | 280 | 77 | 262.35 | 2020.1 |
| V27 | 292.5 | 101 | 260.33 | 2629.3 |
| V28 | 245 | 283 | 408.62 | 11563.9 |
| V29 | 275 | 398 | 408.62 | 16263.1 |
| V30 | 245 | 98 | 362.55 | 3553.0 |
| V31 | 229 | 586 | 430.54 | 25229.6 |
| V32 | 294 | 1088 | 482.62 | 52509.1 |
| V33 | 309.6 | 418 | 396.53 | 16575.0 |
| V34 | 275.6 | 588 | 434.66 | 25558.0 |
| V35 | 309 | 350 | 464.69 | 16264.2 |
| V36 | 331 | 1167 | 542.67 | 63329.6 |
| V37 | 274 | 641.5 | 366.50 | 23510.9 |

TABLE XI
Results of A1% 1 cm and of molar absorptivity in 305 nm (λ) from
the substances V1 to V19

| Substance | A1%1 cm | PM (g/mol) | ε |
|---|---|---|---|
| V1 | 27.5 | 318.19 | 875.0 |
| V2 | 25.3 | 407.21 | 1030.2 |
| V3 | 10.2 | 320.19 | 326.6 |
| V4 | 9.6 | 409.21 | 392.8 |
| V5 | 9.1 | 302.2 | 275.0 |
| V6 | 15.6 | 344.21 | 537.0 |
| V7 | 9.6 | 304.20 | 292.0 |
| V8 | 8.8 | 346.21 | 304.7 |
| V9 | 71.4 | 346.19 | 2471.8 |
| V10 | 37.3 | 388.20 | 1448.0 |
| V11 | 11.6 | 374.21 | 434.1 |
| V12 | 97.7 | 348.19 | 3401..8 |
| V13 | 83.3 | 390.20 | 3250.4 |
| V14 | 9.3 | 376.21 | 349.9 |
| V15 | 82 | 334.05 | 2739.2 |
| V16 | 75.5 | 338.52 | 2555.8 |
| V17 | 15.9 | 304.43 | 484.0 |
| V19 | 8.9 | 318.54 | 283.5 |

TABLE XII
Results of A1% 1 cm and of molar absorptivity in 305 nm (λ) from
the substances V20 to V37

| Substance | A1%1 cm | PM (g/mol) | ε |
|---|---|---|---|
| V20 | 86.8 | 423.66 | 3677.4 |
| V21 | 6.6 | 361.59 | 238.6 |
| V23 | 48.4 | 408.61 | 1977.7 |
| V24 | 62.7 | 374.56 | 2348.5 |
| V25 | 10 | 376.58 | 376.6 |
| V26 | 7.7 | 262.35 | 202.0 |
| V27 | 75.7 | 260.33 | 1970.7 |
| V28 | 137.5 | 408.62 | 5618.5 |
| V29 | 54.8 | 408.62 | 2239.2 |
| V30 | 65.1 | 362.55 | 2360.2 |
| V31 | 0 | 430.54 | 0.0 |
| V32 | 983.8 | 482.62 | 47480.2 |
| V33 | 465.6 | 396.53 | 18462.4 |
| V34 | 140.2 | 434.66 | 6093.9 |
| V35 | 339.6 | 464.69 | 15780.9 |
| V36 | 746.8 | 542.67 | 40526.6 |
| V37 | 95.8 | 366.5 | 3511.07 |

In the 320 nm wavelength (table XIII and XIV), that it is the end of the UVB area and beginning of the UVA area, the most effective substances were 32, 33, 35 and 36. In the UVA area (320-400 nm), represented by the 350 nm wavelength (tables XV and XVI), the substance V36 demonstrated to be very effective in the absorption of this radiation.

TABLE XIII
Results of A1% 1 cm and of molar absorptivity in 320 nm (λ) from
the substances V1 to V19

| Substance | A1%1 cm | PM (g/mol) | ε |
|---|---|---|---|
| V1 | 262 | 318.19 | 8336.6 |
| V2 | 24.6 | 407.21 | 1001.7 |
| V3 | 9.7 | 320.19 | 310.6 |
| V4 | 9 | 409.21 | 368.3 |
| V5 | 9 | 302.20 | 272.0 |
| V6 | 13.5 | 344.21 | 464.7 |
| V7 | 9 | 304.20 | 273.8 |
| V8 | 8.6 | 346.21 | 297.7 |
| V9 | 47.7 | 346.19 | 1651.3 |
| V10 | 24.7 | 388.20 | 958.9 |
| V11 | 8.1 | 374.21 | 303.1 |
| V12 | 72.6 | 348.19 | 2527.9 |
| V13 | 59.1 | 390.20 | 2306.1 |
| V14 | 6.5 | 376.21 | 244.5 |
| V15 | 55.6 | 334.05 | 1857.3 |
| V16 | 51.1 | 338.52 | 1729.8 |
| V17 | 14.5 | 304.43 | 441.4 |
| V19 | 8.6 | 318.54 | 273.9 |

TABLE XIV
Results of A1% 1 cm and of molar absorptivity in 320 nm (λ) from
the substances V20 to V37

| Substance | A1%1 cm | PM (g/mol) | ε |
|---|---|---|---|
| V20 | 44 | 423.66 | 1864.1 |
| V21 | 6.3 | 361.59 | 227.8 |
| V23 | 46.6 | 408.61 | 1904.1 |
| V24 | 16.2 | 374.56 | 606.8 |
| V25 | 6.7 | 376.58 | 252.3 |
| V26 | 5.9 | 262.35 | 154.8 |
| V27 | 17.4 | 260.33 | 453.0 |
| V28 | 61.2 | 408.62 | 2500.8 |
| V29 | 106.2 | 408.62 | 4339.5 |
| V30 | 63.7 | 362.55 | 2309.4 |
| V31 | 0.1 | 430.54 | 4.3 |
| V32 | 403.6 | 482.62 | 19478.5 |
| V33 | 384.2 | 396.53 | 15234.7 |
| V34 | 9.8 | 434.66 | 426.0 |
| V35 | 261 | 464.69 | 12128.4 |
| V36 | 1088.4 | 542.67 | 59064.2 |
| V37 | 10 | 366.50 | 366.5 |

TABLE XV
Results of A1% 1 cm and of molar absorptivity in 350 nm (λ) from
the substances V1 to V19

| Substance | A1%1 cm | PM (g/mol) | ε |
|---|---|---|---|
| V1 | 23.7 | 318.19 | 754.1 |
| V2 | 22.9 | 407.21 | 932.5 |
| V3 | 7.9 | 320.19 | 253.0 |
| V4 | 6.6 | 409.21 | 270.1 |
| V5 | 6.5 | 302.20 | 196.4 |
| V6 | 9.5 | 344.21 | 327.0 |
| V7 | 7.3 | 304.20 | 222.1 |
| V8 | 7.0 | 346.21 | 242.3 |
| V9 | 10.3 | 346.19 | 356.6 |
| V10 | 6.9 | 388.20 | 267.9 |
| V11 | 6.4 | 374.21 | 239.5 |
| V12 | 7.9 | 348.19 | 275.1 |
| V13 | 7.8 | 390.20 | 304.4 |
| V14 | 5.7 | 376.21 | 214.4 |
| V15 | 12.9 | 334.05 | 430.9 |
| V16 | 10.9 | 338.52 | 369.0 |
| V17 | 10.1 | 304.43 | 307.5 |
| V19 | 6.3 | 318.54 | 200.7 |

TABLE XVI
Results of A1% 1 cm and of molar absorptivity in 350 nm (λ) from
the substances V20 to V37

| Substance | A1%1 cm | PM (g/mol) | ε |
|---|---|---|---|
| V20 | 14.7 | 423.66 | 622.8 |
| V21 | 5.7 | 361.59 | 206.1 |
| V23 | 17.9 | 408.61 | 731.4 |
| V24 | 7.8 | 374.56 | 292.2 |
| V25 | 5.7 | 376.58 | 214.7 |
| V26 | 5.3 | 262.35 | 139.0 |
| V27 | 5.2 | 260.33 | 135.4 |
| V28 | 19.8 | 408.62 | 809.1 |
| V29 | 120 | 408.62 | 4903.4 |
| V30 | 7.2 | 362.55 | 261.0 |
| V31 | 0 | 430.54 | 0.0 |
| V32 | 1.4 | 482.62 | 67.6 |
| V33 | 6.3 | 396.53 | 249.8 |
| V34 | 0.8 | 434.66 | 34.8 |
| V35 | 0 | 464.69 | 0.0 |
| V36 | 831 | 542.67 | 45095.9 |
| V37 | 0.6 | 366.5 | 21.9 |

### EXAMPLE 20

### In Vitro SPF

The determination of in vitro SPF has as main principle the spectrophotometry. This method uses the mathematical equation developed by Mansur (Mansur, J. S., Breder, M. N. R., Mansur, M. C. A. et al., 1986, An. Bras. Dermatol., Rio de Janeiro, 61 (3), 121-24):

$$F P S espectrofotom {e}^{′} trico = FC \cdot {\sum }_{320}^{290} E E ( λ ) \cdot I ( λ ) \cdot Abs ( λ )$$

This test results are presented in tables XVII and XVIII.

TABLE XVII
Results of the sun protection factors (SPF) from the
substances V1 to V19 in the respective concentrations,
using ethanol as solvent.

| Substance | Concentration | SPF |
|---|---|---|
| V1 | 4 g % | 0.064 |
| V2 | 5 g % | 0.45 |
| V3 | 1 g % | 0.025 |
| V4 | 1 g % | 0.029 |
| V5 | 5 g % | 0.047 |
| V6 | 5 g % | 0.061 |
| V7 | 5 g % | 0.027 |
| V8 | 5 g % | 0 |
| V9 | 5 g % | 0.61 |
| V10 | 5 g % | 0.39 |
| V11 | 4 g % | 0.093 |
| V12 | 5 g % | 0.93 |
| V13 | 5 g % | 0.66 |
| V14 | 5 g % | 0.72 |
| V15 | 5 g % | 0.74 |
| V16 | 1 g % | 0.15 |
| V17 | 1 g % | 0 |
| V19 | 5 g % | 0 |

TABLE XVIII
Results of the sun protection factors (SPF) from the
substances V20 to V37 in a 5% concentration, with the respective
solvents.

| Substance | SPF | Solvent |
|---|---|---|
| V20 | 0.9 | Ethanol |
| V21 | 0 | Ethanol |
| V23 | 0.53 | Ethanol |
| V24 | 0.52 | Ethanol |
| V25 | 0.1 | Ethanol |
| V26 | 0.059 | Ethanol |
| V27 | 0.76 | Ethanol |
| V28 | 0.74 | Ethanol |
| V29 | 0.9 | THF |
| V30 | 0.5 | Ethanol |
| V31 | 0 | Ethanol |
| V32 | 9.5 | Chloroform |
| V33 | 5.2 | Ethanol |
| V34 | 1.7 | Hexane |
| V35 | 3.3 | THF |
| V36 | 7.7 | DMSO |
| V37 | 1.1 | THF |

The substances V32, V33, V34, V35, V36 and V37 presented the best SPF values. While the CNSL directly derived substances (V1-V19) and others synthesized from it (V20-V21) presented almost null SPF values.

1-24. (canceled)

25. Composition for the photoprotection of surfaces characterized by comprising at least one compound of formula (I) 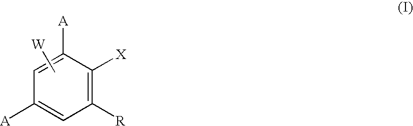
where R is alkyl, alkenil, octyl, pentadecyl, 1-[(E)-1-pentadecenyl, 1-[(Z)-8-pentadecenyl, 1-[(8Z,11Z)-8,11-pentadecadienyl, 1-[(8Z,11Z)-8,11,14-pentadecatrienyl, cycloalkyl, alkoxyl, B-alkoxyl, B-sulfanyl, B-sulfonyl, B-sulfinyl, B-sulfonates, B-sulfonamides, B-amino, B-carbamoyl, B-halides, B-carboalkoxyl, B-carbothioalkoxyl, N,N-B-carbamoyl, B-trihaloalkane, B-ciano, nitro-B, azido-B, B-amines, B-amides, halides, carboalkoxyl, carbothioalkoxyl, N,N-dissubstituted-carbamoyl, trihaloalkane, ciano, nitro, azido or C₈OR₂;
B is hydrogen, alkyl, alkenyl, cicloalkyl, cicloalkenyl or aryl;
X is hydrogen, carboxyl, alkylcarboxyl, alkenylcarboxyl, alkylcarboxylate, alkenylcarboxylate, carbothioate, carbodithioate, carboalkoxyl, carbamoyl, formyl, alkylcarbonyl, arylcarbonyl, (E)-2-propenoic acid, (2E,4E)-2,4-pentadienoic acid, sulfonic acid, (E)-1-ethene-1-sulphonic, (1E,3E)-1,3-butadiene-1-sulfonic acid and its homo-derivated or its alkylic, phenolic, benzylic or cinnamic esters, lactones, amides, lactames and imides, W-benzoyl;
A is hydrogen or R₁;
R₁ is hydrogen, hydroxyl, alkyl, cycloalkyl; phenyl, furyl, thiophenyl, pyridinyl, pyrimidinyl, pyrrolyl, thiazolyl, W-quinazolyl, W-isoquinolyl, W-benzimidazolyl, W-benzoxazolyl, W-benzothiazolyl, acyl, acetyl, W-cinnamoyl, chrotyl, W-benzoyl, alkoxyl, cycloalkoxyl, B-alkoxyl, B-sulfanyl, B-sulfonyl, B-sulfinyl, B-sulfonates, B-sulfonamides, B-amino, B-carbamoyl, B-halides, B-carboalkoxyl, B-carbothioalkoxyl, N,N-B-carbamoyl, B-trihaloalkane, B-ciano, nitro-B, azido-B, N,N-di-B-carbamoyl, trihaloalkane;
B is hydrogen, alkyl, alkenyl, cicloalkyl, cicloalkenyl or aryl;
W is hydrogen, ortho-hydroxyl, ortho-alkyl, ortho-cycloalkyl, ortho-alkoxyl, ortho-cycloalkoxyl, ortho-sulfanyl, ortho-aryloxyl, ortho-sulfones, ortho-sulfides, ortho-sulfinyl, ortho-sulfonates, ortho-sulfonamides, ortho-amine, ortho-amide, ortho-halides, ortho-carboalkoxyl, ortho-carbothioalkoxyl, ortho-carbamoyl, ortho-trihaloalkane, ortho-ciano, ortho-nitro, ortho-acyl, ortho-acetyl, ortho-benzoyl, ortho-4-alkyloxybenzoyl, ortho-4-alkoxybenzoyl ortho-4-methoxybenzoyl, ortho-4-dimethylaminobenzoyl, ortho-cinnamoyl, ortho-4-alkyloxycinnamoyl, ortho-4-methoxycinnamoyl, ortho-3-(4-methoxyphenyl)-3-oxo-propanoyl, ortho-3-(4-alkoxyphenyl)-3-oxo-propanoyl, ortho-3-(4-phenoxyphenyl)-3-oxo-propanoyl, ortho-3-(4-aminophenyl)-3-oxo-propanoyl, ortho-3-(4-carbamoylphenyl)-3-oxo-propanoyl, ortho-3-(4-methoxyphenyl)-1,3-propanodione, ortho-3-(4-alkoxyphenyl)-1,3-propanodione, ortho-3-(4-phenoxyphenyl)-1,3-propanodione, ortho-3-(4-aminophenyl)-1,3-propanodione, ortho-3-(4-carbamoylphenyl)-1,3-propanodione, ortho-2H-benzo[d][1,2,3]triazol-2-yl, meta-hydroxyl, meta-alkyl, meta-cycloalkyl, meta-alkoxyl, meta-cycloalkoxyl, meta-sulfanyl, meta-aryloxyl, meta-sulfones, meta-sulfides, meta-sulfinyl, meta-sulfonates, meta-sulfonamides, meta-amine, meta-amide, meta-halides, meta-carboalkoxyl, meta-carbothioalkoxyl, meta-carbamoyl, meta-trihaloalkane, meta-ciano, meta-nitro, meta-acyl, meta-acetyl, meta-benzoyl, meta-4-alkyloxybenzoyl, meta-4-alkoxybenzoyl, meta-4-methoxybenzoyl, meta-4-dimethyilaminobenzoyl, meta-cinnamoyl, meta-4-alkyloxycinnamoyl, meta-4-methoxycinnamoyl, meta-3-(4-methoxyphenyl)-3-oxo-propanoyl, meta-3-(4-alkoxyphenyl)-3-oxo-propanoyl, meta-3-(4-phenoxyphenyl)-3-oxo-propanoyl, meta-3-(4-aminophenyl)-3-oxo-propanoyl, meta-3-(4-carbamoylphenyl)-3-oxo-propanoyl, meta-3-(4-methoxyphenyl)-1,3-propanodione, meta-3-(4-alkoxyphenyl)-1,3-propanodione, meta-3-(4-phenoxyphenyl)-1,3-propanodione, meta-3-(4-aminophenyl)-1,3-propanodione, meta-3-(4-carbamoylphenyl)-1,3-propanodione, meta-2H-benzo[d][1,2,3]triazol-2-yl, para-hydroxyl, para-alkyl, para-cycloalkyl, para-alkoxyl, para-cycloalkoxyl, para-sulfanyl, para-aryloxyl, para-sulfones, para-sulfides, para-sulfinyl, para-sulfonates, para-sulfonamides, para-amine, para-amide, para-halides, para-carboalkoxyl, para-carbothioalkoxyl, para-carbamoyl, para-trihaloalkane, para-ciano, para-nitro, para-acyl, para-acetyl, para-benzoyl, para-4-alkyloxybenzoyl, para-4-alkoxybenzoyl, para-4-methoxybenzoyl, para-4-dimethyilaminobenzoyl, para-cinnamoyl, para-alkyloxycinnamoyl or para-4-methoxycinnamoyl, para-3-(4-methoxyphenyl)-3-oxo-propanoyl, para-3-(4-alkoxyphenyl)-3-oxo-propanoyl, para-3-(4-phenoxyphenyl)-3-oxo-propanoyl, para-3-(4-aminophenyl)-3-oxo-propanoyl, para-3-(4-carbamoylphenyl)-3-oxo-propanoyl, para-3-(4-methoxyphenyl)-1,3-propanodione, para-3-(4-alkoxyphenyl)-1,3-propanodione, para-3-(4-phenoxyphenyl)-1,3-propanodione, para-3-(4-aminophenyl)-1,3-propanodione, para-3-(4-carbamoylphenyl)-1,3-propanodione, para-2H-benzo[d][1,2,3]triazol-2-yl;
R₂ is hydrogen, hydroxyl, alkyl, alkenyl, alkinyl, cycloalkyl, cycloalkoxyl, B-alkoxyl, B-sulfanyl, B-sulfonyl, B-sulfinyl, B-sulfonates, B-sulfonamides, B-amino, B-carbamoyl, B-halides, B-carboalkoxyl, B-carbothioalkoxyl, N,N-B-carbamoyl, B-trihaloalkane, B-ciano, nitro-B, azido-B, alkoxyl, phenyl, furyl, thiophenyl, pyridinyl, pyrimidinyl, pyrrolyl, thiazolyl, W-quinazolyl, W-isoquinolyl, W-benzimidazolyl, W-benzoxazolyl, W-benzothiazolyl, acyl, acetyl, W-cinnamoyl, chrotyl, W-benzoyl, N,N-di-B-carbamoyl, trihaloalkane;
B is hydrogen, alkyl, alkenyl, cicloalkyl, cicloalkenyl or aryl;
with the proviso that:
when R is 1-[(8Z,11Z)-8,11,14-pentadecatrienyl, X is hydrogen, alkylcarboxyl, alkenylcarboxyl, alkylcarboxylate, alkenylcarboxylate, carbothioate, carbodithioate, carboalkoxyl, carbamoyl, formyl, alkylcarbonyl, arylcarbonyl, (E)-2-propenoic acid, (2E,4E)-2,4-pentadienoic acid, sulfonic acid, (E)-1-ethene-1-sulphonic, (1E,3E)-1,3-butadiene-1-sulfonic acid and its homo-derivated or its alkylic, phenolic, benzylic or cinnamic esters, lactones, amides, lactames and imides, W-benzoyl; and
when X and R forms a 6-membered heterocyclic ring, X is adjacent to an oxygen atom and R is C₁-C₈ alkyl optionally substituted with one carbonyl, hydroxyl, thiol, halide or amine; C₁-C₈ alkenyl optionally substituted with a carbonyl, hydroxyl, thiol, halide or amine; 8-(1-octanol), 8-(E)-7-octen-1-ol, 8-(E)-6-ceto-7-octen-1-ol, 8-(1-octanethiol), 8-(E)-7-octene-1-thiol, 8-(E)-6-ceto-7-octene-1-thiol, 8-(1-octanamine), 8-(E)-7-octen-1-amine, 8-(E)-6-ceto-7-octen-1-amine.

26. The composition according to claim 25, wherein the surface to be protected comprise at least one of the skin, hair and nails.

27. The composition according to claim 25, wherein the surface to be protected is selected from the group that comprises furniture, equipments, industrial surfaces, residential surfaces, automobiles, plastic surfaces, wood surfaces and combination of the referred surfaces.

28. The composition according to claim 25, wherein the referred composition is selected from the group that comprises at least one of inks, varnishes and similar coverings, at least one of plastic compositions and a mix of them, at least one of cosmetic, pharmaceutical products and mixtures thereof.

29. The use of a compound characterized by formula (I) 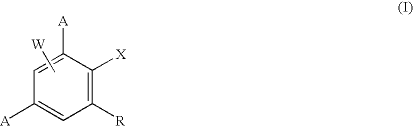
where R is alkyl, alkenil, octyl, pentadecyl, 1-[(E)-1-pentadecenyl, 1-[(Z)-8-pentadecenyl, 1-[(8Z,11Z)-8,11-pentadecadienyl, 1-[(8Z,11Z)-8,11,14-pentadecatrienyl, cycloalkyl, alkoxyl, B-alkoxyl, B-sulfanyl, B-sulfonyl, B-sulfinyl, B-sulfonates, B-sulfonamides, B-amino, B-carbamoyl, B-halides, B-carboalkoxyl, B-carbothioalkoxyl, N,N-B-carbamoyl, B-trihaloalkane, B-ciano, nitro-B, azido-B, B-amines, B-amides, halides, carboalkoxyl, carbothioalkoxyl, N,N-dissubstituted-carbamoyl, trihaloalkane, ciano, nitro, azido or C₈OR₂
B is hydrogen, alkyl, alkenyl, cicloalkyl, cicloalkenyl or aryl;
X is hydrogen, carboxyl, alkylcarboxyl, alkenylcarboxyl, alkylcarboxylate, alkenylcarboxylate, carbothioate, carbodithioate, carboalkoxyl, carbamoyl, formyl, alkylcarbonyl, arylcarbonyl, (E)-2-propenoic acid, (2E,4E)-2,4-pentadienoic acid, sulfonic acid, (E)-1-ethene-1-sulphonic, (1E,3E)-1,3-butadiene-1-sulfonic acid and its homo-derivated or its alkylic, phenolic, benzylic or cinnamic esters, lactones, amides, lactames and imides, W-benzoyl;
A is hydrogen or R₁;
R₁ is hydrogen, hydroxyl, alkyl, cycloalkyl; phenyl, furyl, thiophenyl, pyridinyl, pyrimidinyl, pyrrolyl, thiazolyl, W-quinazolyl, W-isoquinolyl, W-benzimidazolyl, W-benzoxazolyl, W-benzothiazolyl, acyl, acetyl, W-cinnamoyl, chrotyl, W-benzoyl, alkoxyl, cycloalkoxyl, B-alkoxyl, B-sulfanyl, B-sulfonyl, B-sulfinyl, B-sulfonates, B-sulfonamides, B-amino, B-carbamoyl, B-halides, B-carboalkoxyl, B-carbothioalkoxyl, N,N-B-carbamoyl, B-trihaloalkane, B-ciano, nitro-B, azido-B, N,N-di-B-carbamoyl, trihaloalkane;
B is hydrogen, alkyl, alkenyl, cicloalkyl, cicloalkenyl or aryl;
W is hydrogen, ortho-hydroxyl, ortho-alkyl, ortho-cycloalkyl, ortho-alkoxyl, ortho-cycloalkoxyl, ortho-sulfanyl, ortho-aryloxyl, ortho-sulfones, ortho-sulfides, ortho-sulfinyl, ortho-sulfonates, ortho-sulfonamides, ortho-amine, ortho-amide, ortho-halides, ortho-carboalkoxyl, ortho-carbothioalkoxyl, ortho-carbamoyl, ortho-trihaloalkane, ortho-ciano, ortho-nitro, ortho-acyl, ortho-acetyl, ortho-benzoyl, ortho-4-alkyloxybenzoyl, ortho-4-alkoxybenzoyl ortho-4-methoxybenzoyl, ortho-4-dimethylaminobenzoyl, ortho-cinnamoyl, ortho-4-alkyloxycinnamoyl, ortho-4-methoxycinnamoyl, ortho-3-(4-methoxyphenyl)-3-oxo-propanoyl, ortho-3-(4-alkoxyphenyl)-3-oxo-propanoyl, ortho-3-(4-phenoxyphenyl)-3-oxo-propanoyl, ortho-3-(4-aminophenyl)-3-oxo-propanoyl, ortho-3-(4-carbamoylphenyl)-3-oxo-propanoyl, ortho-3-(4-methoxyphenyl)-1,3-propanodione, ortho-3-(4-alkoxyphenyl)-1,3-propanodione, ortho-3-(4-phenoxyphenyl)-1,3-propanodione, ortho-3-(4-aminophenyl)-1,3-propanodione, ortho-3-(4-carbamoylphenyl)-1,3-propanodione, ortho-2H-benzo[d][1,2,3]triazol-2-yl, meta-hydroxyl, meta-alkyl, meta-cycloalkyl, meta-alkoxyl, meta-cycloalkoxyl, meta-sulfanyl, meta-aryloxyl, meta-sulfones, meta-sulfides, meta-sulfinyl, meta-sulfonates, meta-sulfonamides, meta-amine, meta-amide, meta-halides, meta-carboalkoxyl, meta-carbothioalkoxyl, meta-carbamoyl, meta-trihaloalkane, meta-ciano, meta-nitro, meta-acyl, meta-acetyl, meta-benzoyl, meta-4-alkyloxybenzoyl, meta-4-alkoxybenzoyl, meta-4-methoxybenzoyl, meta-4-dimethyilaminobenzoyl, meta-cinnamoyl, meta-4-alkyloxycinnamoyl, meta-4-methoxycinnamoyl, meta-3-(4-methoxyphenyl)-3-oxo-propanoyl, meta-3-(4-alkoxyphenyl)-3-oxo-propanoyl, meta-3-(4-phenoxyphenyl)-3-oxo-propanoyl, meta-3-(4-aminophenyl)-3-oxo-propanoyl, meta-3-(4-carbamoylphenyl)-3-oxo-propanoyl, meta-3-(4-methoxyphenyl)-1,3-propanodione, meta-3-(4-alkoxyphenyl)-1,3-propanodione, meta-3-(4-phenoxyphenyl)-1,3-propanodione, meta-3-(4-aminophenyl)-1,3-propanodione, meta-3-(4-carbamoylphenyl)-1,3-propanodione, meta-2H-benzo[d][1,2,3]triazol-2-yl, para-hydroxyl, para-alkyl, para-cycloalkyl, para-alkoxyl, para-cycloalkoxyl, para-sulfanyl, para-aryloxyl, para-sulfones, para-sulfides, para-sulfinyl, para-sulfonates, para-sulfonamides, para-amine, para-amide, para-halides, para-carboalkoxyl, para-carbothioalkoxyl, para-carbamoyl, para-trihaloalkane, para-ciano, para-nitro, para-acyl, para-acetyl, para-benzoyl, para-4-alkyloxybenzoyl, para-4-alkoxybenzoyl, para-4-methoxybenzoyl, para-4-dimethyilaminobenzoyl, para-cinnamoyl, para-alkyloxycinnamoyl or para-4-methoxycinnamoyl, para-3-(4-methoxyphenyl)-3-oxo-propanoyl, para-3-(4-alkoxyphenyl)-3-oxo-propanoyl, para-3-(4-phenoxyphenyl)-3-oxo-propanoyl, para-3-(4-aminophenyl)-3-oxo-propanoyl, para-3-(4-carbamoylphenyl)-3-oxo-propanoyl, para-3-(4-methoxyphenyl)-1,3-propanodione, para-3-(4-alkoxyphenyl)-1,3-propanodione, para-3-(4-phenoxyphenyl)-1,3-propanodione, para-3-(4-aminophenyl)-1,3-propanodione, para-3-(4-carbamoylphenyl)-1,3-propanodione, para-2H-benzo[d][1,2,3]triazol-2-yl;
R₂ is hydrogen, hydroxyl, alkyl, alkenyl, alkinyl, cycloalkyl, cycloalkoxyl, B-alkoxyl, B-sulfanyl, B-sulfonyl, B-sulfinyl, B-sulfonates, B-sulfonamides, B-amino, B-carbamoyl, B-halides, B-carboalkoxyl, B-carbothioalkoxyl, N,N-B-carbamoyl, B-trihaloalkane, B-ciano, nitro-B, azido-B, alkoxyl, phenyl, furyl, thiophenyl, pyridinyl, pyrimidinyl, pyrrolyl, thiazolyl, W-quinazolyl, W-isoquinolyl, W-benzimidazolyl, W-benzoxazolyl, W-benzothiazolyl, acyl, acetyl, W-cinnamoyl, chrotyl, W-benzoyl, N,N-di-B-carbamoyl, trihaloalkane; and
B is hydrogen, alkyl, alkenyl, cicloalkyl, cicloalkenyl or aryl;
with the proviso that:
when R is 1-[(8Z,11Z)-8,11,14-pentadecatrienyl, X is hydrogen, alkylcarboxyl, alkenylcarboxyl, alkylcarboxylate, alkenylcarboxylate, carbothioate, carbodithioate, carboalkoxyl, carbamoyl, formyl, alkylcarbonyl, arylcarbonyl, (E)-2-propenoic acid, (2E,4E)-2,4-pentadienoic acid, sulfonic acid, (E)-1-ethene-1-sulphonic, (1E,3E)-1,3-butadiene-1-sulfonic acid and its homo-derivated or its alkylic, phenolic, benzylic or cinnamic esters, lactones, amides, lactames and imides, W-benzoyl; and
when X and R forms a 6-membered heterocyclic ring, X is adjacent to an oxygen atom and R is C₁-C₈ alkyl optionally substituted with one carbonyl, hydroxyl, thiol, halide or amine; C₁-C₈ alkenyl optionally substituted with a carbonyl, hydroxyl, thiol, halide or amine; 8-(1-octanol), 8-(E)-7-octen-1-ol, 8-(E)-6-ceto-7-octen-1-ol, 8-(1-octanethiol), 8-(E)-7-octene-1-thiol, 8-(E)-6-ceto-7-octene-1-thiol, 8-(1-octanamine), 8-(E)-7-octen-1-amine, 8-(E)-6-ceto-7-octen-1-amine,
in the preparation of compositions capable of absorbing ultraviolet radiation.

30. The use according to claim 29, wherein said compound absorbs radiation in the wavelength range from about 200 nm to about 400 nm.

31. The use according to claim 29, wherein said compound absorbs radiation in the wavelength range from about 200 nm to about 280 nm.

32. The use according to claim 29, wherein said compound absorbs radiation in the wavelength range from about 280 nm to about 320 nm.

33. The use according to claim 29, wherein said compound absorbs radiation in the wavelength range from about 320 nm to about 400 nm.

34. The use according to claim 29, wherein said compound absorbs radiation in the wavelength range from about 280 nm to about 400 nm.
